(19) United States
(12) Patent Application Publication
(10) Pub. No.: US 2022/0249490 A1
(43) Pub. Date: Aug. 11, 2022

(54) GENE THERAPY FOR MESOTHELIOMA

(71) Applicant: The Trustees of the University of Pennsylvania, Philadelphia, PA (US)

(72) Inventors: Daniel Sterman, Philadelphia, PA (US); Steven Albelda, Philadelphia, PA (US)

(21) Appl. No.: 17/505,364

(22) Filed: Oct. 19, 2021

Related U.S. Application Data
(62) Division of application No. 16/456,066, filed on Jun. 28, 2019, now abandoned.
Application No. 16/456,066 is a division of application No. 15/672,330, filed on Aug. 9, 2017, now abandoned.
(60) Provisional application No. 62/304,233, filed on Mar. 6, 2016.

(51) Int. Cl. A61K 31/519; A61K 45/06; A61P 35/00; A61K 31/415; A61K 31/7068; A61K 35/761; C12N 15/86; C07K 14/47; A61K 38/21; A61K 31/635; A61K 31/555; A61K 33/243; A61K 9/00; A61K 39/12; A61K 39/395

(52) U.S. Cl. CPC A61K 31/519; C12N 2710/10343; A61P 35/00; A61K 31/415; A61K 31/7068; A61K 35/761; C12N 15/86; C07K 14/4703; A61K 38/212; A61K 31/635; A61K 31/555; A61K 33/243; A61K 9/0019; A61K 38/21; A61K 39/12; A61K 39/3955; A61K 45/06

(57) ABSTRACT

Gene therapy based combination therapy for malignant pleural mesothelioma (“MPM”) that is resistant to or recurrent after chemotherapy employing a viral vector containing a human interferon transgene, followed by standard first- or second-line cytotoxic chemotherapy. Overall survival rate was significantly higher than historical controls in the second-line group.

### RELATED APPLICATIONS

This application is a divisional of co-pending U.S. Ser. No. 15/672,330 filed 9 Aug. 2017, which in turn asserts priority from Patent Cooperation Treaty application PCT/US2017/020856 filed 6 Mar. 2017, which in turn asserts priority from U.S. provisional patent filing Ser. No. 62/304,233 filed 6 Mar. 2016, the contents of which are here incorporated by reference.

### GOVERNMENT INTEREST

This work was funded in part by National Cancer Institute grant No. NCI P01 CA66726.

### RESEARCH AGREEMENTS

This work was sponsored by FKD Therapies Limited, manufacturer of the rAd-IFN immune-gene vector used here.

### BRIEF DESCRIPTION

“In situ vaccination” using immuno-gene therapy has the ability to induce polyclonal anti-tumor responses directed by the patient's immune system.

Experimental Design: Human patients with un-resectable malignant pleural mesothelioma (MPM) received two intra-pleural doses of a replication-defective adenoviral vector containing the human interferon-alpha (hIFN-α2b) gene (Ad.IFN) concomitant with a 14-day course of a cyclooxygenase-2 inhibitor (celecoxib), followed by standard first- or second-line cytotoxic chemotherapy. Primary outcomes were safety, toxicity, and objective response rate; secondary outcomes included progression-free and overall survival. Bio-correlates on blood and tumor were measured.

Results: Forty subjects, ECOG PS 0 or 1, were treated: 18 received first-line pemetrexed-based chemotherapy with platinum, 22 received second-line chemotherapy with pemetrexed (n=7) or a gemcitabine-based regimen (n=15). Treatment was well tolerated and adverse events were comparable to historical controls. Using Modified RECIST, the overall response rate was 25% and the disease control rate was 88%. Median overall survival (MOS) for all patients with epithelial histology was 21 months (95% CI [12,∞]) versus 7 months for patients with non-epithelial histology (95% CI [6,∞]). MOS in the first-line chemotherapy cohort was 12.5 months (95% CI [8,21]), while MOS for the second-line chemotherapy cohort was 21.5 months (95% CI [9,∞]), with 32% of patients alive at 2 years. No biologic parameters were found to correlate with response, including numbers of activated blood T cells or NK cells, number of regulatory T cells in blood, peak levels of interferon-α in blood or pleural fluid, induction of anti-tumor antibodies, nor an immune-gene signature in pretreatment biopsies.

Conclusions: The combination of intrapleural Ad.IFN, celecoxib, and systemic chemotherapy proved safe in patients with MPM. Overall survival rate was significantly higher than historical controls in the second-line group. Results of this study support proceeding with a multi-center randomized clinical trial of chemo-immunogene therapy versus standard chemotherapy alone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows response to Ad.IFN plus chemotherapy in a waterfall plot of radiographic responses. p FIG. 2 shows response to Ad.IFN plus chemotherapy in a spider plot using the percent change in tumor size as assessed from modified RECIST measurements.

FIG. 3 shows response to Ad.IFN plus chemotherapy in a spider plot using the fold change in the serum mesothelin reactive protein (SMRP) for non-responders.

FIG. 4 shows response to Ad.IFN plus chemotherapy in a spider plot using the fold change in the serum mesothelin reactive protein (SMRP) for responders. p Response rates using modified RECIST1.1 are shown in FIG. 1. FIGS. 2 and 3 show the changes in modified RECIST measurements and serum mesothelin (SMRP) levels respectively compared to baseline.

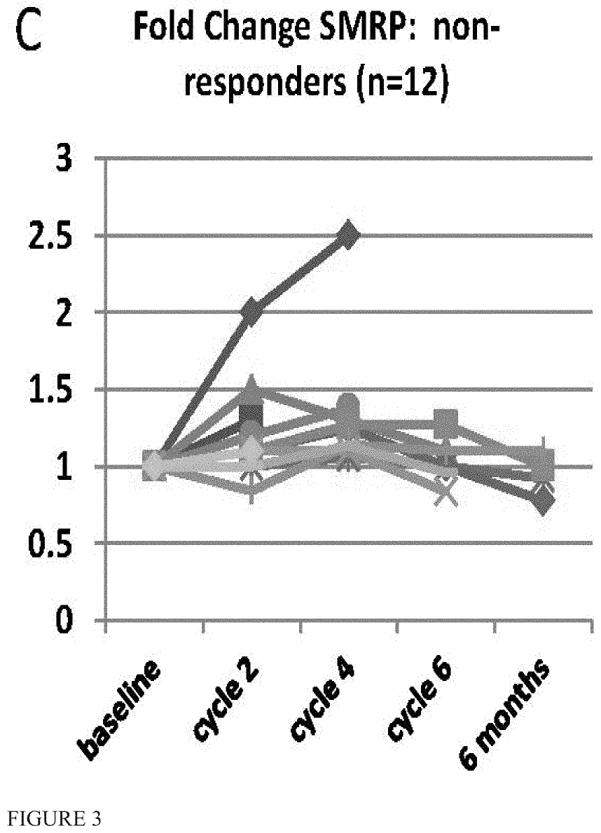

FIGS. 5 shows Kaplan-Meier plots for survival for all subjects (n=40) (A) or subjects segregated by tumor histology (non-epithelial (n=10) versus epithelial (n=30)) (B), subjects receiving first-line therapy with pemetrexed(n=18) (C) , subjects receiving second-line therapy (n=22) (D), and second-line subjects segregated by type of chemo (gemcitabine based (n=15) versus pemetrexed based (n=7) (E). FIG. 5 shows the Kaplan-Meier curve of the entire patient group. A number of subgroups were analyzed.

FIG. 6 shows a significant (log rank, p=0.004) difference in MOS for the 30 patients with epithelial histology (19 months) versus the 10 patients with non-epithelial histology (6.5 months).

FIG. 7 shows the 18 treatment-naïve patients treated with front line-line chemotherapy had a MOS of 12 months (95% CI [6,15]) with a median PFS of 6.5 months (95% CI [5.5,11.5]).

FIG. 8 shows survival in the 22 patients treated with second-line therapy. The MOS for the second-line cohort was 17 months (95% CI [6.5,26]).

FIG. 9 is a subgroup analysis of the second-line cohort.

FIGS. 10-12 show MPM specimens ranked for intensity of expression of immune response-related genes. Tracks are (left to right) MN, OK6, REN 208, 213, 302, 307, M30, M60; molecular weight markers at 64, 51, 39, 28, 14 and 9.7 kD.

FIG. 10A shows results for patient #424 at Day 1. FIG. 10B shows results at Day 57.

FIG. 11A shows results for patient #430 at Day 1. FIG. 1B shows results at Day 57.

FIG. 12A shows results for patient #422 at Day 1. FIG. 12B shows results at Day 57.

FIG. 13 shows the distribution of baseline adenoviral Nab titers.

FIG. 14 shows serum levels of interferon-a measured pre-vector infusion (Day 1).

FIG. 15 shows levels of IFNα in the pleural fluid or the pleural lavage measured at baseline in 38 patients.

FIG. 16 shows correlation of survival times (all patients) with the serum interferon level.

FIG. 17 shows correlation of survival times (epithelial patients) with the serum interferon level.

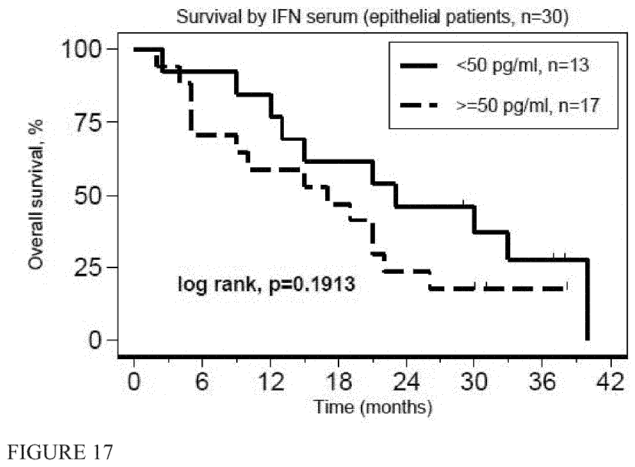

FIG. 18 shows correlation of survival times (all patients) with the pleural interferon level.

FIG. 19 shows correlation of survival times (epithelial patients) with the pleural interferon level

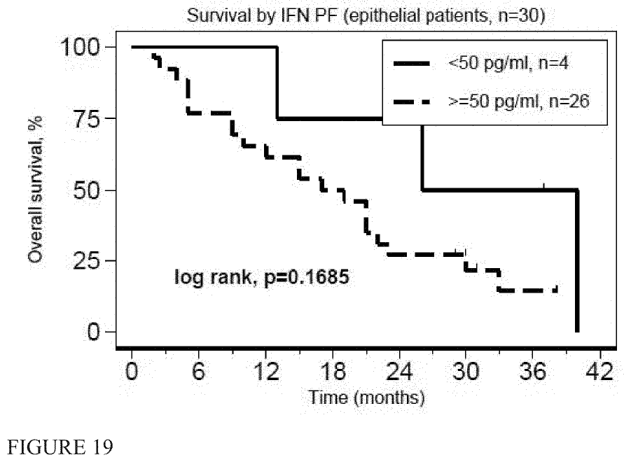

FIG. 20 shows immunohistochemistry correlations with the degree of lymphocyte (CD8 staining), infiltration (all patients).

FIG. 21 shows immunohistochemistry correlations with the degree of lymphocyte (CD8 staining) infiltration (epithelial patients).

FIG. 22 shows immunohistochemistry correlations with the degree of macrophage (CD68 staining) infiltration (all patients).

FIG. 23 shows immunohistochemistry correlations with the degree of macrophage (CD68 staining) infiltration (epithelial patients).

FIG. 24 shows immunohistochemistry correlations with the degree of expression of PD-L1 (all patients).

FIG. 25 shows immunohistochemistry correlations with the degree of expression of PD-L1 (epithelial patients).

FIG. 26 shows immunohistochemistry correlations with the degree of expression of PD-L1 0-1 vs 2-5 (all patients).

FIG. 27 shows immunohistochemistry correlations with the degree of expression of PD-L1 0-1 vs 2-5 (epithelial patients).

FIG. 28 shows survival by immune scores (all patients).

FIG. 29 shows survival by immune scores (epithelial patients).

Table 1 summarizes Patient demographics for forty patients with MPM enrolled on the trial between March 2011 and October 2013.

Table 2A shows serious adverse events.

Table 2B shows adverse events during the chemotherapy portion of the study.

Table 3 shows response rates using modified RECIST1.1.

Table 4 shows the median overall survival (MOS) for current front-line standard-of-care chemotherapy regimen of pemetrexed and cisplatin (or carboplatin).

Table 5 shows subsequent therapies and effects.

Table 6 shows flow cytometry from PBMC in 6 patients who had good responses (average survival=23.5 months) and compared results to 6 patients with poor responses (average survival=7.2 months).

Table 7 shows RNA interrogated for 600 immune response-related genes using NANOSTRING® technology.

Table 8 shows expression of anti-tumor antibodies in the serum of post-treatment patients.

Table 9 shows increases in the NK activation receptors NKp46, NKG2D, NKG2A and NKp30 and changes in a CD8 T cell activation signature (CD38hi/HLA-DRhi and ki67hi/Bcl -2low).

### DETAILED DESCRIPTION

Malignant pleural mesothelioma (MPM) is a rapidly progressive thoracic neoplasm with high mortality that typically responds poorly to standard medical regimens (1). The current front-line standard-of-care chemotherapy regimen is pemetrexed and cisplatin (or carboplatin), resulting in a median overall survival (MOS) of 12-13 months (Table 4).

TABLE 4
Results of some recent trials of chemotherapy in MPM

| | | | | Response | TTP/ | Median | 1 Yr | |
|---|---|---|---|---|---|---|---|---|
| | | | | rate | PFS | OS | OS | |
| Reference | Type | % Epi | # Pts | (%) | (mo) | (mo) | (%) | DCR |
| Zucali [15] | prior pem/cis retreat | 90 | 59 | 15 | 2.3 | 6.2 | 25 | 52 |
| | with vinorelbine | | | | | | (est) | |
| | Patient Status: Naive | | | | | | | |
| Vogelzang [1] | pem/cis | 68 | 168 | 46 | 6.1 | 13.3 | 57 | |
| Van | ralitrexed/cis | 75 | 126 | 24 | 5.3 | 11.4 | 46 | |
| Meerbeeck [2] | | | | | | | | |
| Lee [3] | carbo/pem | 37 | NR | NR | 10 | 41 | | |
| | Patient Status: Pre- | | | | | | | |
| | Treated | | | | | | | |
| Zauder [17] | Pem/cis retreated with | 67 | 60 | 2 | 1.7 | 5.2 | NR | 48 |
| | Gem or Venorelbine | | | | | | | |
| Ceresoli [4] | carbo/pem | 78 | 102 | 19 | 6.5 | 12.7 | 51 | 78 |
| Nowack [18] | Pem/cis treated with | 67 | 30 | 3 | 1.5 | 8.2 | 45 | 46 |
| | BNC105 | | | | | | (est) | |
| Krug [5] | Pem/Cis | 30 | 10 | 3.4 | 12.8 | 50 | 60 | 30 |
| | | | | | | (est) | | |
| Santoro [6] | pem/cis (Int. expanded | 67 | 745 | 26 | 7 | not | 63 | 78 |
| | access trial) | | | | | avail | | |
| | pem/carbo (Int. expanded | 67 | 752 | 22 | 6.9 | not | 64 | 76 |
| | access trial) | | | | | avail | | |
| | Average | 71 | | 25 | 5.9 | 12 | 53 | 71 |
| | current trial | 61 | 18 | 28 | 6.5 | 12 | 55 | 83 |
| Hassan [7] | Meso Ab and pem cis | 89 | 89 | 40 | 6.1 | 14.8 | 60 | 91 |
| Zucali [8] | prior pem, then gem or | | | 30 | 10 | 2.8 | 10.9 | NR |
| | vinorelbine | | | | | | | |
| Xanthopoulos | prior pem, | | 29 | 22 | 2.0 | 6.0 | NR | |
| [9] | then -oxali/gem | | | | | | | |
| Stebbing [10] | vinorelbine | | 62 | 63 | 16 | | 9.6 | NR |
| Pasello [11] | carbo/gem | | 17 | NR | 3.6 | 6.6 | NR | |
| Dubey [12] | sorafenib | | 50 | NR | 3.6 | 9.7 | NR | |
| Margery [13] | pem or gem/oxali | | 44 | NR | 3.8 | 12.2 | NR | |
| Ceresoli [14] | re-treat with pem | | 31 | 19 | 3.8 | 10.5 | NR | |
| Zucali [16] | review 2L (all) | | 75 | 181 | 11 | 4.3 | 8.7 | 34 |
| Zucali [16] | Pem retreated with Pem | | 42 | ? | 6.5 | 11 | 50 | 65 |
| Calabro [19] | Pem/cis treated with | 86 | 29 | 7 | 6.2 | 10.7 | 48 | 31 |
| | tremilumimab | | | | | | | |
| Average | | | | 11 | 3.5 | 9.0 | 44 | 52 |
| Current Trial | All 2nd line | | 22 | 14 | 4.0 | 17 | 60 | 91 |
| Zucali [16] | review 2L (all) | 75 | 181 | 11 | 4.3 | 8.7 | 34 | 71 |
| Current Trial | All 2nd line | | 22 | 14 | 4.0 | 17 | 60 | 91 |
| Zucali [16] | Pem retreated with Pem | | 42 | ? | 6.5 | 11 | 50 | 65 |
| Ceresoli [14] | Pe, m re-treat with pem | | 31 | 19 | 3.8 | 10.5 | NR | |
| Bearz | Pem, re-treat with pem | | 30 | 17 | 5.1 | 13.6 | 50 | 67 |
| | | | | | | | (2 yr- | |
| | | | | | | | 30%) | |
| Average | | | | 18 | 5.1 | 11.7 | 50 | 66 |
| Current Trial | Prior Pem- repeat Pem | | 7 | 28 | 8.0 | 26 | 86 | 86 |

References
[1] Vogelzang N J, Rusthoven J J, Symanowski J, et al. Phase III study of pemetrexed in combination with cisplatin versus cisplatin alone in patients with malignant pleural mesothelioma. Journal of Clinical Oncology 2003; 21: 2636-2644.
[2] van Meerbeeck J P, Gaafar R, Manegold C, et al. Randomized phase III study of cisplatin with or without raltitrexed in patients with malignant pleural mesothelioma: an intergroup study of the European Organisation for Research and Treatment of Cancer Lung Cancer Group and the National Cancer Institute of Canada. Journal of Clinical Oncology 2005; 23: 6881-6889.
[3] Lee C W, Murray N, Anderson H, Rao S C, Bishop W. Outcomes with first-line platinum-based combination chemotherapy for malignant pleural mesothelioma: a review of practice in British Columbia. Lung Cancer 2009; 64: 308-313.
[4] Ceresoli G L, Zucali P A, Favaretto A G, et al. Phase II study of pemetrexed plus carboplatin in malignant pleural mesothelioma. Journal of Clinical Oncology 2006; 24: 1443-1448.
[5] Krug L M, Wozniak A J, Kindler H, et al. Randomized phase II trial of pemetrexed/cisplatin with or without CBP501 in patients with advanced malignant pleural mesothelioma, Lung Cancer, 2014; 85: 429-34
[6] Santoro A, O'Brien M E, Stahel R A, et al. Pemetrexed plus cisplatin or pemetrexed plus carboplatin for chemonaïve patients with malignant pleural mesothelioma: results of the International Expanded Access Program. J Thorac Oncol. 2008; 3: 756-63.
[7] Hassan R, Kindler H L, Jahan T, et al. Phase 2 Trial of Amatuximab, a chimeric antimesothelin antibody with pemetrexed and cisplatin in advance unresectable pleural mesothelioma, Clin Can Res 2014; 20: 5927-36.
[8] Zucali P A, Ceresoli G L, Garassino I, et al. Gemcitabine and vinorelbine in pemetrexed-pretreated patients with malignant pleural mesothelioma. Cancer 2008; 112: 1555-61.
[9] Xanthopoulos A, Bauer T T, Blum T G, Kollmeier J, Schonfeld N, Serke M. Gemcitabine combined with oxaliplatin in pretreated patients with malignant pleural mesothelioma: an observational study. Journal of Occupational Medicine & Toxicology 2008; 3: 34.
[10] Stebbing J, Powles T, McPherson K, et al. The efficacy and safety of weekly vinorelbine in relapsed malignant pleural mesothelioma. Lung Cancer 2009; 63: 94-97.
[11] Pasello G, Nicotra S, Marulli G, et al. Platinum-based doublet chemotherapy in pre-treated malignant pleural mesothelioma (MPM) patients: A mono-institutional experience. Lung Cancer 2011; 73: 351-5.
[12] Dubey S, Janne P A, Krug L, Pang H, Wang X, Heinze R, et al. A phase II study of sorafenib in malignant mesothelioma: results of Cancer and Leukemia Group B 30307. Journal of Thoracic Oncology 2010; 5: 1655-1661.
[13] Margery J, Riviere F, Planchard D, Le Floch H, Ferrand F R, Mairovitz A, et al. [Second-line therapy in patients with malignant pleural mesothelioma. A French retrospective study (2005-2006)]. Revue de Pneumologie Clinique 2010; 66: 255-259.
[14] Ceresoli G L, Zucali P A, De Vincenzo F, et al. Retreatment with pemetrexed-based chemotherapy in patients with malignant pleural mesothelioma. Lung Cancer 2011; 72: 73-77.
[15] Zucali P A, Perrino M, Lorenzi E, et al., Vinorelbine in pemetrexed-pretreated patients with malignant pleural mesothelioma. Lung Cancer 2014; 84: 265-70
[16] Zucali P A, Simonelli M, Michetti G, et al., Second-line chemotherapy in malignant pleural mesothelioma: Results of a retrospective multicenter survey. Lung Cancer. 2012; 75: 360-7.
[17] Zauderer M G, Kass S L, Woo K, Sima C S, Ginsberg M S, Krug L M. Vinorelbine and gemcitabine as second- or third-line therapy for malignant pleural mesothelioma. Lung Cancer. 2014; 84: 271-4.
[18] Nowak A K, Brown C, Millward M J, et al., A phase II clinical trial of the Vascular Disrupting Agent BNC105P as second line chemotherapy for advanced Malignant Pleural Mesothelioma. Lung Cancer 2013; 81: 422-427
[19] Calabrò L, Morra A, Fonsatti E, et al. Tremelimumab for patients with chemotherapy-resistant advanced malignant mesothelioma: an open-label, single-arm, phase 2 trial. Lancet Oncol 2013; 14: 1104-11
20. Bearz A, Talamini2 R, Rossoni, G. Re-challenge with pemetrexed in advanced mesothelioma: a multi-institutional experience. BMC Research Notes 2012, 5: 482

Patients with progressive disease may be offered additional agents, including drugs such as gemcitabine or vinorelbine, but second-line treatments for MPM have not demonstrated significant response rates or improvements in survival, and have not been approved by the FDA for this indication (1, 2). For patients with MPM receiving second-line chemotherapy, the MOS is approximately 9 months (Table 4).

Given these suboptimal results, our group has explored the use of in situ immuno-gene therapy to treat MPM using first-generation, replication-deficient adenoviruses (Ad) administered intrapleurally (3). Our recent work focused on Ad vectors encoding type 1 interferon genes (initially interferon-β, then subsequently interferon-α) (4-6). Although type 1 interferons have been used with some success in certain tumors (7) and intrapleural interferon-gamma showed some efficacy in early stage mesothelioma (8), the high doses required and associated systemic side effects have limited the utility of this approach, a problem potentially overcome by localized delivery of cytokine genes.

After intrapleural injection, Ad.IFN efficiently transfects both benign mesothelial and malignant mesothelioma cells, resulting in the production of large concentrations of interferon within the pleural space and tumor (4-6). Mesothelioma cell transduction with Ad.IFN results in tumor cell death and a powerful stimulus to the immune system, as type 1 interferons augment tumor neo-antigen presentation/processing in dendritic cells, induce TH1 polarization, and augment cytotoxic CD8+ T cell function, as well as that of NK cells, and M1 phenotype macrophages (7,9). The inflammatory response to the Ad viral vector itself also elicits additional “danger signals,” further potentiating anti-tumor immune responses (10). This multi-pronged approach alters the tumor microenvironment, kills tumor cells, and stimulates the innate and adaptive immune systems.

We previously showed safety, feasibility, and induction of anti-tumor humoral and cellular immune responses in Phase I intrapleural Ad.IFN trials (4-6). We also identified a maximally-tolerated dose and demonstrated that two doses of Ad.IFN-alpha-2b administered with a dose interval of 3 days resulted in augmented gene transfer without enhanced toxicity. In some patients, this approach appeared to “break tolerance”—engendering a long-lasting response (presumably immunologic) characterized by tumor regression at distant sites over months without further therapy. A trial using the same Ad.IFN-alpha-2b vector via intravesical instillation in bladder cancer patients has also demonstrated promising results (11).

Although encouraging, the percentage and degree of tumor responses in our Phase 1 studies were limited. We attempted to augment the efficacy of adenoviral immuno-gene therapy in preclinical models by adding cyclooxygenase-2 inhibition (mitigating the immunosuppressive tumor microenvironment by decreasing PGE2 and IL-10 production) (12) and by concomitant/adjuvant administration of chemotherapy (13). This latter approach fits well with the emerging consensus that immune stimulation by certain forms of chemotherapy—by exposure of tumor neo-antigens to dendritic cells and depletion of regulatory T cells, among other mechanisms—is crucial to therapeutic efficacy (14-17). Accordingly, we designed a pilot and feasibility study in MPM patients who were not candidates for surgical resection to assess the safety and activity of two doses of intrapleural Ad.hIFN-α2b (given in combination with high dose celecoxib) followed by standard first-line or second-line chemotherapy.

### Methods

### Study Design and Patients

In this single-center, open-label, non-randomized pilot and feasibility trial, there were two primary outcome measures: 1) safety and toxicity, and 2) tumor response (by Modified RECIST). Secondary outcomes included PFS, OS, and bio-correlates of clinical response and multiple immunologic parameters.

The vector used in this trial, originally called SCH 721015 (Ad.hIFN-α2b), is a clinical-grade, serotype 5, E1/partial E3-deleted replication-incompetent adenovirus with insertion of the human IFN-α2b gene in the E1 region of the adenoviral genome (6). It was provided by the Schering-Plough Research Institute (Kenilworth, N.J.).

Eligibility stipulated: [1] pathologically-confirmed MPM; [2] ECOG performance status of 0 or 1; and [3] accessible pleural space for vector instillation. Exclusion criteria included pericardial effusion, inadequate pulmonary function (FEV1 <1 liter or <40% of predicted value (post-pleural drainage)), significant cardiac, hepatic, or renal disease, or high neutralizing anti-Ad antibody (Nabs) titers (>1:2000).

The stopping criteria and detailed description of adverse events that served as dose limiting toxicities (DLTs) is described in the Supplemental Methods. Very briefly, DLTs were defined (using NIC criteria) by any Grade 4 toxicity, Grade 3 hypotension or allergic reaction, Grade 3 non-hematologic toxicity persisting for more than 7 days, persistent cytokine release syndrome, or Grade 3 hematologic toxicity persisting for >7 days.

The protocol was approved by the Penn IRB (UPCC 02510), the FDA (BB-IND 13854), and the NIH Recombinant DNA Advisory Committee. Written informed consent was obtained from patients at the time of screening, and the study was registered at clinicaltrials.nih.gov (NCT01119664).

### Study Design

Eligible MPM patients underwent tunneled intrapleural catheter insertion under local anesthesia or via thoracoscopy (6). On Study Days 1 and 4, a dose of 3×10¹¹ viral particles (vp) Ad.hIFN-α2b, diluted in 25-50 cc of sterile normal saline, was instilled into the pleural space. Patients were observed in the Clinical and Translational Research Center (CTRC) of the University of Pennsylvania Medical Center for at least 24 hours after vector instillation. The vector was administered concomitant with a 14-day course of oral celecoxib (400 mg twice daily starting three days prior to vector instillation).

Fourteen days after the first dose of vector, patients initiated outpatient chemotherapy in one of two treatment groups: Treatment-naïve patients received standard-dose front-line chemotherapy with pemetrexed and a platinum agent (either cisplatin or carboplatin). Those undergoing second-line chemotherapy primarily received gemcitabine+/−carboplatin (Table 1).

TABLE 1
Basic Demographics and Patient Characteristics

| Patients | n = 40 | |
|---|---|---|
| Age in years | Avg (Median) 68 (67) | |
| Male Gender | 29 (72%) | |
| Cancer Stage | I | 3 (8%) |
| | II | 3 (8%) |
| | III | 16 (40%) |
| | IV | 18 (45%) |
| Histologic Type | Epithelial | 30 (75%) |
| | Biphasic | 5 (12.5%) |
| | Sarcomatoid | 4 (10%) |
| | Lympho-histocytic | 1 (2.5%) |
| Type of | 1^{st} Line Pemetrexed/platin | 18 (45%) |
| Chemotherapy | 2^{nd} Line Repeat Pemetrexed/platin | 7 (17.5%) |
| | 2^{nd} Line GEmcitabine +/− platin | 15 (37.5%) |

In addition, the second-line cohort included patients who had undergone pemetrexed-based chemotherapy at least 6 months previously with disease stability or response. These subjects were retreated with pemetrexed, as has been reported in the medical literature (Table 4).

Patients were monitored as outpatients through Day 190, and thereafter by telephone or electronic medical record. Patients were assessed for anti-tumor responses every 6 weeks after initial treatment using chest CT scans up until 6 months. If progression was documented at the initial follow-up CT scan (approximately 2 months post vector dosing), then subjects proceeded with other therapeutic options, but continued to be followed (Table 5).

TABLE 5
Post-Trial Therapies

| | | | | 1st |
|---|---|---|---|---|
| | | | | Line |
| | | | | or 2nd |
| ID | Post Dosing Treatment | | | Line |
| 402 | Navelbine × 1 course | | | 2 |
| 404 | TGF-beta, CIR T cells, Pem X 1 cycle | | | 2 |
| 405 | Liver immunoembolization @ TJUH | | | 2 |
| 406 | Gem maintenance | | | 1 |
| 407 | Radiation | | | 2 |
| 408 | 06511-GEM +/− NGR-hTNF × 2 | | | 1 |
| 409 | None | | | 2 |
| 410 | None | | | 1 |
| 411 | CIR T cells, PEMX 3, Rad, Consented for | | | 2 |
| | AdV-tk trial Dose #2(#2 patient), palliative | | | |
| | RT cervical nodale mass | | | |
| 412 | None | | | 1 |
| 413 | None | | | 1 |
| 416 | Rad, SBRT February 2013, Pem(March 2013- | | | 2 |
| | October 2013) | | | |
| 417 | Gem maintenance May 2012- | 2 | Pem maintenance; | 1 |
| | June 2014, Cytoreductive surgery, | | AdV-tk trial Dose | |
| | tremelimumab study | | Level #2 | |
| 418 | Maintenance PEM/Gem October 2013-August 2014, Pem | | | 2 |
| | September 2014 | | | |
| 419 | Palliative Rad for pain | | | 1 |
| 420 | Recent progression-treatment with PEM. | | | 2 |
| 421 | None | | | 2 |
| 422 | None | | | 2 |
| 423 | Palliative RT | | | 1 |
| 424 | Palliative RT-CTCA/Home Hospice | | | 1 |
| 425 | Maintenance PEM/PD on 2/1013 CT GEM started | | | 2 |
| 426 | Home Hospice | | | 1 |
| 427 | UPCC 06511/gem +/− NGR-hTNFX2 treatments | | | 1 |
| 428 | Rad to pleural cath site. Hospice | | | 1 |
| 429 | PEM maintenance | | | 1 |
| 430 | Gem maintenance; AdV-tk(L2), | 2 | Pem maintenance/ | 2 |
| | palliative radiation with | 431 | Proton Tx to pleurx | |
| | hyperthermia | | site; T cell meso | |
| | | | trial, Lilly | |

on Dec. 2, 2013(#1)-GEM, Palliative RT, Navelbine

| | | |
|---|---|---|
| 432 | PEM maintenance | 1 |
| 433 | PEM/carbo, palliative radiation, Pem/carboX3 C, PEM, | 2 |
| | GEM locally, Considering T cell | |
| 434 | None | 2 |
| 435 | Navelbine locally-started April 2013 | 2 |
| 436 | Pleurectomy/PDT-May 20, 2014/Carbo-Alimta X6 | 1 |
| | cycles, ?proton tx | |
| 437 | C30901-Randomized to Pem maintenance | 1 |
| | (versus observation) | |
| 438 | Pem maintenance, Gem, Tremelilumimab vs. Placebo, | 2 |
| | Navelbine | |
| 439 | Rad to pleural cath site/Pem maintenance | 1 |
| | | 441 |

trial LY3023414

| | | |
|---|---|---|
| 442 | Tremelimumab study-Duke(January 2014-April 2014) | 2 |
| | Navelbine locally | |
| 443 | None | 1 |
| 445 | Palliative Rad txp | 2 |
| 446 | Tremimulimab trial at Duke | 2 |

After 6 months, patients were tracked in return visits, by communications with local physicians, and by phone conversations. Times of death and progression were recorded; subsequent treatments and the causes of death were determined where possible. Radiographic analysis was performed by a board-certified thoracic radiologist (SK) blinded to the patients' medical history and other clinical trial results. Modified RECIST measurements were recorded at each exam (18).

### Biocorrelates

Enzyme-linked immunosorbent assays (ELISAs) were used to measure IFN-α2b levels (PBL Biomedical Labs; Piscataway, N.J.), as well as serum mesothelin-related protein (SMRP) levels (Fujirebio, Inc., Malvern, Pa.). Neutralizing adenovirus antibody titers (Nabs) were assessed as previously described (5). To detect induced humoral responses against tumor antigens, we performed immunoblotting against purified mesothelin and extracts from allogeneic mesothelioma cell lines using pre- and post-treatment serum as previously described (4-6). See Supplemental Methods for details.

Cryopreserved peripheral blood mononuclear cells (PBMC) were collected prior to treatment, 2 days after Ad.IFN instillation (before the second dose) and 15 days after the first dose (just prior to chemotherapy administration). PBMCs were studied from a set of six patients who responded to therapy and 6 patients who progressed with treatment (Table 6).

TABLE 6
Characteristics of Patients Selected for Flow Cytometry Analysis

| | | survival | ave | | | | | |
|---|---|---|---|---|---|---|---|---|
| Patient | Line | (November | survival | | RECIST | ave | Antibody | type of |
| Number | of Rx | 2013) | (mo) | selection code | Response | response | Response | chemo |
| 404 | 2 | 15 | | responder | −7% | | 0 | G |
| 411 | 2 | 33 | | responder | −6% | | 0 | G |
| 416 | 2 | 25 | | responder | −12% | | 0 | G |
| 406 | 1 | 21 | | responder | −48% | | 1 | P |
| 405 | 1 | 40 | | responder | −32% | | 2 | P |
| 417 | 2 | 31+ | | responder | 13% | | 2 | G |
| | | | 26.8 | | | −15.3% | | |
| 425 | 2 | 9 | | non-responder | −19% | | 1 | P2 |
| 402 | 2 | 6.5 | | non-responder | 10% | | 2 | G |
| 421 | 2 | 5 | | non-responder | 5% | | 2 | G |
| 422 | 2 | 2.5 | | non responder | 80% | | 2 | P2 |
| 429 | 1 | 9 | | non responder | −14% | | 1 | P1 |
| 423 | 1 | 11 | | non responder | −4% | | 1 | P1 |
| | | | 7.2 | | | 9.7% | | |

Antibody Code: 0 = no change, 1 = marginal changes, 2 = significant changes.

PBMC were thawed and the activation of natural killer cell (NK) and T cells was assessed using flow cytometry as detailed in the Supplemental Methods (see also Ref 19).

Formalin-fixed paraffin-embedded sections from original surgical biopsies or previous surgery were available from 18 patients and stained with anti-CD8, anti-CD68, or anti-PDL1 antibodies. Tissue sections were also assessed for RNA levels using Nanostring® analysis. (see Supplemental Methods for details).

Immuno-Gene Score

To evaluate the basal “immune activation” state of the tumors, we adapted the recently described “immunoscore” derived from studies used to predict immune responses of melanoma and lung cancer patients to an anti-cancer MAGE vaccine (20). This study identified 84 genes (mostly related to CD8 T cells and interferon responses) that correlated with response. We had information on 27 of the 61 PCR-validated genes in our nanostring data (see Table 7).

TABLE 7
List of Immune Response
Genes assayed by Nanostring
CCL5
CD3D
CD86
CD8a
CDC42SE1
CXCL10
CXCL2
CXCL9
EPSTI1
GBP1
GCH1
GZMK
ICOS
IL2RG
IRF1
ITK
KLRD1
PSMB8
PSMB9
PTGER4
SLAMF6
SLAMF7
STAT1
TARP
TNFAIP3
TNFRSF9
TOX

The sum of the intensity of each of these 27 genes was determined and each tumor ranked from highest expression to lowest expression.

Statistical Analysis

Our original Penn IRB approval was for enrollment of 10-15 patients in each of the two cohorts: first and second-line chemotherapy. With a minimum of 11 patients in a treatment stratum, we had 90% power to identify any unanticipated toxicity with prevalence of >19%; We were ultimately provided with enough vector to treat 40 patients, so we subsequently received IRB approval for a study amendment allowing for a total number of 40 patients, allowing us to treat 18 first-line and 22 second-line patients. This provided us with 90% power to identify any unanticipated toxicity with prevalence of ˜12%.

Efficacy was determined by estimating objective response rates and distributions of times to progression and death. We summarized the distributions of PFS and OS by Kaplan-Meier curves, comparing curves across strata by the log rank test.

Statistics used for the flow cytometry data are described in the Supplementary

### Methods.

### Results

Forty patients with MPM were enrolled on the trial between March 2011 and October 2013. Patient demographics are summarized in Table 1.

Thirty-two patients received two intrapleural doses of Ad.hIFN-α2b. Eight patients received only one dose of vector because of: 1) low serum albumin (n=1); 2) shortness of breath (n=2); 3) increased serum transaminases (n=1); 4) supraventricular tachycardia (n=1); or 5) decreased absolute neutrophil count (n=3). In several of the 8 cases wherein patients received a single dose and were ineligible for repeat dosing, the adverse effects that precluded repeat dosing were at least in part attributable to expected adverse events secondary to the initial vector dose.

All 40 patients were able to begin chemotherapy treatment 14 days after initial vector instillation. Eighteen of 40 patients (45%) received first-line chemotherapy. Twenty-two patients (55%) received second-line chemotherapy with either pemetrexed (n=7) alone or gemcitabine+/−carboplatin (n=15). At least four cycles of chemotherapy were delivered to all but 10 of the 40 patients. Chemotherapy was stopped in 9 of these 10 patients due to disease progression after one cycle (n=1 [first-line]), two cycles (n=6 [1 first line, 5 second-line]), or three cycles (n=2 [ both second-line]). In the tenth patient, chemotherapy was stopped after one cycle due to development of an acute respiratory decompensation subsequently determined to be unrelated to the protocol.

The study protocol was generally well-tolerated. Most patients experienced only expected mild toxicities from the vector and transgene expression, including cytokine release syndrome, nausea, fatigue, anemia, lymphopenia (grade 3-4), and hypoalbuminemia (Table 2A).

TABLE 2A
Adverse Events Related to Study Treatment

| | GRADE (Number of Events) | | | | |
|---|---|---|---|---|---|
| ADVERSE EVENTS | 1 | 2 | 3 | 4 | TOTAL |
| Syndrome | | | | | |
| Cytokine Release | 14 | 25 | | | 39 |
| Interferon Syndrome* | 9 | 2 | | | 11 |
| Blood | | | | | |
| Hemoglobin-low | 5 | 3 | 2 | | 10 |
| Leukocytes-low | 7 | 4 | | | 11 |
| Lymphopenia | 10 | 11 | 13 | 4 | 38 |
| Neutrophils-low | 5 | 2 | 2 | | 9 |
| Platelets-low | 10 | | | | 10 |
| Cardiac | | | | | |
| Supraventricular | | | 1 | | 1 |
| tachycardia | | | | | |
| Hypertension | 1 | | | | 1 |
| Coagulation | | | | | |
| PTT-high | 4 | 1 | | | 5 |
| Constitutional | | | | | |
| Chills-intermittent | 2 | | | | 2 |
| Fatigue | 2 | | | | 2 |
| Anxiety | 2 | | | | 2 |
| GI | | | | | |
| Nausea | 2 | | | | 2 |
| Anorexia | 2 | 1 | | | 3 |
| Metabolic | | | | | |
| Albumin-low | 19 | 23 | | | 42 |
| ALT-high | 4 | | | | 4 |
| AST-high | 7 | | | | 7 |
| Calcium-low | 22 | 4 | | | 26 |
| Creatinine-high | 2 | 1 | | | 3 |
| Total Bilirubin-high | 1 | 1 | | | 2 |
| Potassium | 2 | | | | 2 |
| Neurology | | | | | |
| Insomnia | 1 | | | | 1 |
| Dizziness | 1 | | | | 1 |
| Pain | | | | | |
| Pleural-post vector | 1 | | | | 1 |
| instillation | | | | | |
| Headache | 1 | | | | 1 |
| Tumor site worsen | 1 | | 1 | | 2 |
| Pulmonary | | | | | |
| Cough | 1 | | | | 1 |
| Atelectasis | | | | | |
| Dyspnea on exertion | 2 | 1 | | | 3 |
| Hypoxia | 1 | | 1 | | 2 |

*= Interferon Syndrome refers to toxicity presumed secondary to interferon production post vector administration similar to side effects of systemic IFN administered for Hep C. Typically, the syndrome is malaise, loss of appetite, mild nausea, and persistent low-grade fevers

These toxicities typically resolved within 24-48 hours of completion of vector dosing, and predominantly occurred after the initial vector infusion. We identified 11 patients who had mild symptoms including temporary malaise, loss of appetite, nausea, and persistent low-grade fevers for a few days after vector instillation, presumably due to systemic interferon effects. Serious adverse events included pleural catheter infection (n=2); hypoxia (n=2); supraventricular tachycardia (SVT) (n=1); and esophagitis (n=1); none was directly attributable to the instillation of the vector (Table 2A). Local infection related to catheter placement was certainly associated with the study protocol, in which the majority of patients underwent catheter insertion specifically for enrollment in this clinical trial, but adverse effects from the catheter were not directly related to the administration of rAdIFN into the pleural space via the catheter or to the rAdIFN vector itself. The one patient with transitory hypoxia experienced a presumed congestive heart failure exacerbation on the day of repeat vector dosing related to planned withholding of diuretics in anticipation of possible hypotension related to vector instillation. The hypoxia rapidly resolved after diuresis. The episode of SVT was seen a single patient with massive tumor burden in the right hemithorax and mediastinum compressing both his left and right atria. The esophagitis was noted in a patient who required stereotactic radiation therapy for palliation of a focal region of her left sided malignant pleural mesothelioma that was compressing her distal esophagus. There were no treatment-related deaths. Adverse events during the chemotherapy portion of the study were expected and comparable to historical controls (Table 2B).

TABLE 2B
Adverse Events Related to Chemotherapy

| | GRADE | | | | |
|---|---|---|---|---|---|
| ADVERSE EVENTS- | (Number of Events) | | | | |
| Chemo Related | 1 | 2 | 3 | 4 | TOTAL |
| Blood | | | | | |
| Hemoglobin-low | 20 | 37 | 10 | 1 | 68 |
| Neutrophils-low | 2 | 1 | 1 | | 4 |
| Lymphopenia | 5 | 1 | 5 | 7 | 18 |
| Neutrophils-low | 2 | | 1 | 2 | 5 |
| Platelets-low | 5 | 1 | 2 | 1 | 9 |
| Leukocytes-low | 6 | 2 | 1 | | 9 |
| Constitutional | | | | | |
| Fatigue | 5 | 5 | | | 10 |
| Fever in absence of | 2 | | | | 2 |
| neutropenia | | | | | |
| Weight-loss | 2 | | | | 2 |
| Weight-increase | 1 | | | | 1 |
| Rigor | 1 | | | | 1 |
| Dermatology | | | | | |
| Alopecia | 1 | | | | 1 |
| Hyperpigmentation-nevi | 1 | | | | 1 |
| Rash-pruritic trunk/UE | | 1 | | | 1 |
| Endocrine | | | | | |
| Cushingoid appearance | | 1 | | | 1 |
| (swelling to face) | | | | | |
| Gastrointestinal | | | | | |
| Anorexia | 3 | 3 | | | 6 |
| Nausea | 10 | 1 | | | 11 |
| Esophagitis | 3 | | 1 | | 4 |
| Diarrhea | 2 | | | | |
| Vomiting | 1 | 1 | | | 2 |
| Hiccoughs | 1 | | | | 1 |
| Metabolic | | | | | |
| Albumin-low | 19 | 23 | | | 42 |
| ALT-high | 1 | 1 | | | 2 |
| AST-high | 1 | 1 | | | 2 |
| Calcium-low | 5 | 2 | 1 | | 8 |
| Sodium-low | 6 | | | | 6 |
| Creatinine | 2 | 1 | | | 3 |
| Potassium | 1 | | | | 1 |
| Neurological | | | | | |
| Dizziness | 2 | | | | 2 |
| Neuropathy | 1 | 1 | | | 2 |
| Tinnitus | | 1 | | | 1 |
| Rhinorrhea/Rhinitis | 2 | | | | 2 |
| Vertigo | 1 | | | | 1 |
| Other: Buzzing in ears | 1 | | | | 1 |
| Other: Numbness hand/feet | 1 | | | | 1 |
| Pain | | | | | |
| Arthralgia | 1 | | | | 1 |
| Tumor site | | 1 | | | 1 |
| Headache | | 1 | | | 1 |
| Pulmonary | | | | | |
| Cough | 1 | | | | 1 |

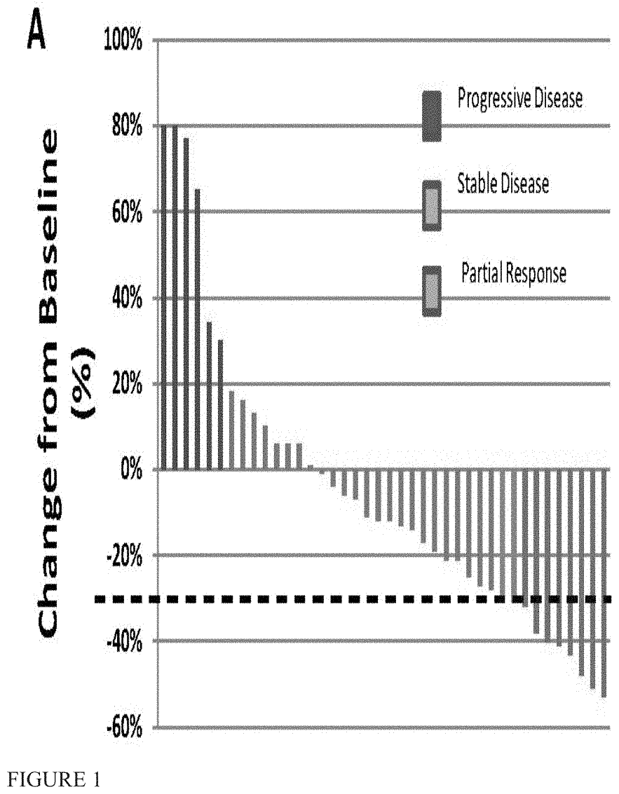

Response rates using modified RECIST1.1 are shown in FIG. 1 and Table 3.

TABLE 3
Responses

| | | | Response | Stable | | Median | Median | OS | OS | OS |
|---|---|---|---|---|---|---|---|---|---|---|
| Patient | | | rate | Disease | DCR | PFS | OS | 1 Yr | 18 mo | 24 mo |
| group | Chemotherapy | # Pts | (%) | % | % | (mo) | (mo) | (%) | % | % |
| All patients | | 40 | 25 | 62.5 | 87.5 | 5.3 | 13 | 55 | 40 | 25 |
| Naïve | Pem/cis | 18 | 28 | 55 | 83 | 6.5 | 12 | 55 | 28 | 17 |
| Pre-treated | All 2nd line | 22 | 14 | 77 | 91 | 4.0 | 17 | 59 | 50 | 32 |
| | Prior Pem- | 7 | 28 | 72 | 100 | 8.0 | 26 | 86 | 86 | 57 |
| | repeat Pem | | | | | | | | | |
| | Prior Pem | 15 | 7 | 80 | 87 | 3.5 | 10 | 47 | 33 | 20 |
| | repeat GEM | | | | | | | | | |

Pem = pemetrexed;
cis = cisplatin;
GEM = gemcitabine

For both cohorts combined, we noted stable disease in 62.5% of patients and partial responses in 25% of patients; no complete responses were observed. Only 12.5% had progressive disease following cycle 2. The overall disease control rate (DCR) was 87.5%. Partial responses were seen in 9/25 (36%) evaluable patients with pemetrexed-based chemotherapy and 1/15 (7%) with gemcitabine-based treatment.

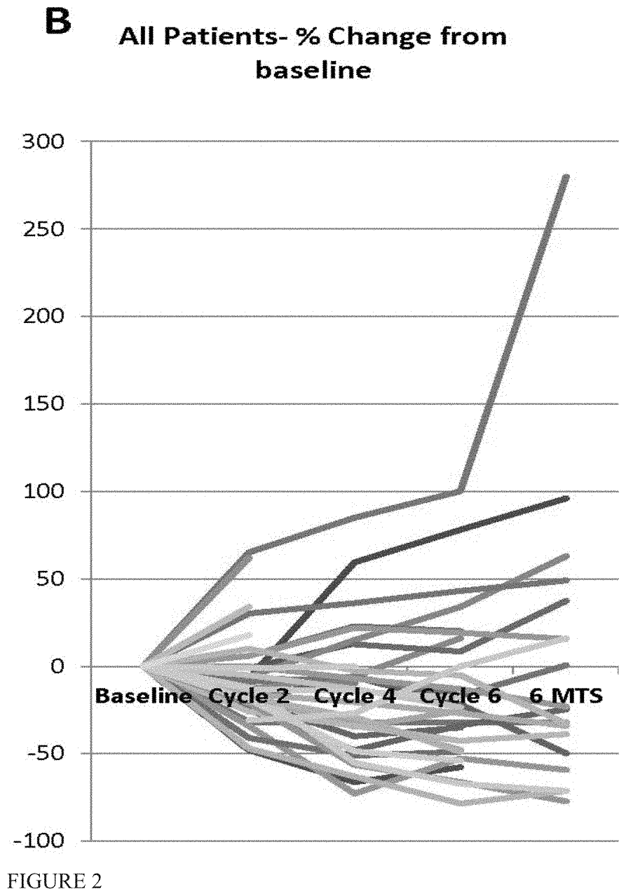

FIGS. 2 and 3 show the changes in modified RECIST measurements and serum mesothelin (SMRP) levels respectively compared to baseline. For SMRP, 12 of the 27 patients showed more than a 20% increase in SMRP level (FIG. 3, upper panel), while 15 of the 27 patients showed a greater than 20% decrease at some time point (FIG. 3 lower panel). Both modified RECIST and SMRP responses were durable.

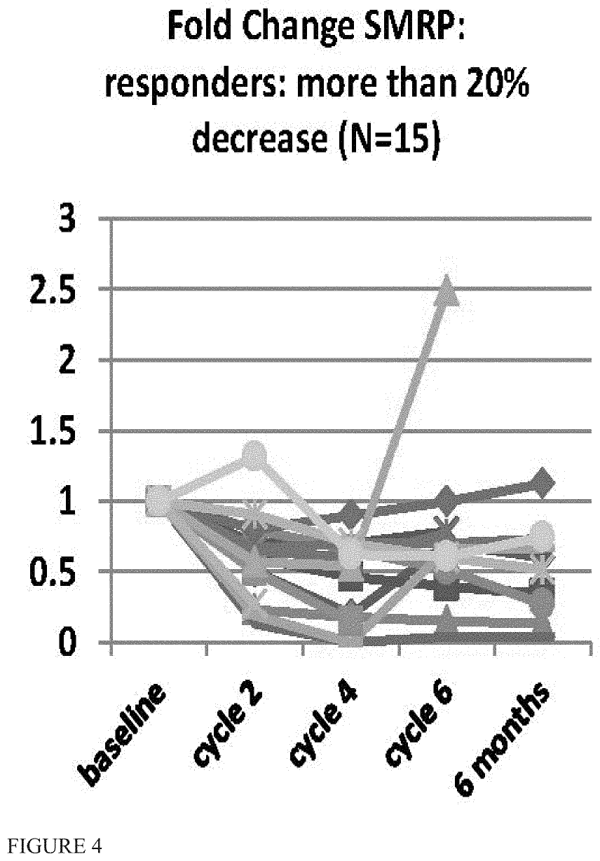

At the time of submission of this manuscript, 6 of 40 patients remained alive with a minimum follow-up of 24 months. All but two of the deceased patients died of progressive disease, with one patient dying from esophageal perforation status post proton-beam radiotherapy (5 months) and another from a BAP-1 deficiency-related metastatic uveal melanoma (40 months). FIG. 5 shows the Kaplan-Meier curve of the entire group. The MOS was 13 months (95% CI [9,12]); however, we noted a significant “tail” to the curve, 341 revealing a subset of patients with prolonged survival. The survival of the entire cohort at 12 months was 55% (95% CI: 0.38, 0.69), at 18 months 40% (95% CI 0.55, 0.25 and at 24 months 25% (95% CI: 0.39, 0.13). The PFS was 5.3 months.

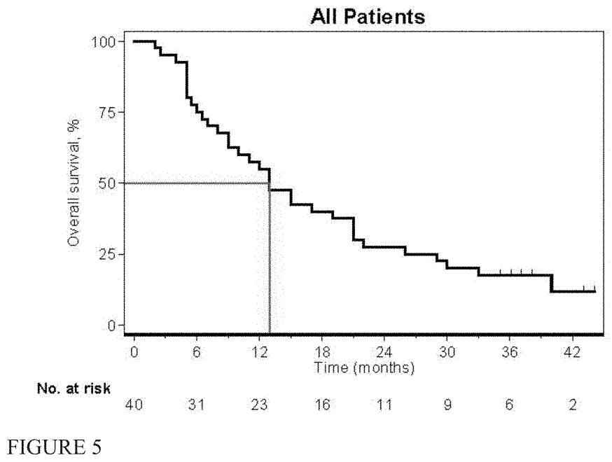

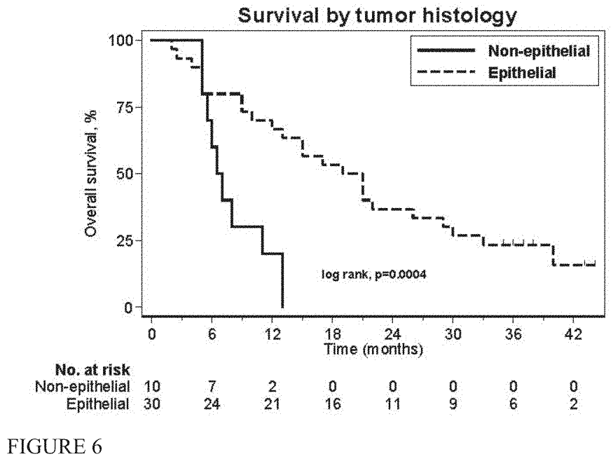

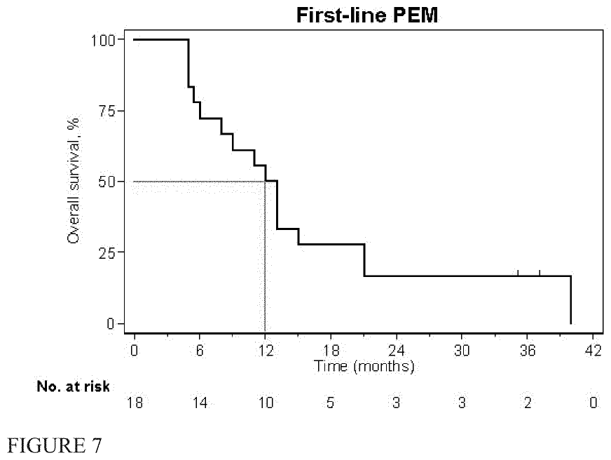

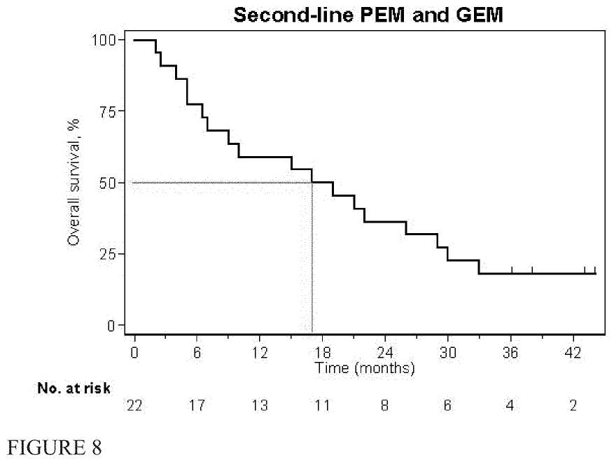

A number of subgroups were analyzed. FIG. 6 shows a significant (log rank, p=0.004) difference in MOS for the 30 patients with epithelial histology (19 months) versus the 10 patients with non-epithelial histology (6.5 months). The 18 treatment-naïve patients treated with front line-line chemotherapy had a MOS of 12 months (95% CI [6,15]) (FIG. 7) with a median PFS of 6.5 months (95% CI [5.5,11.5]). FIG. 8 shows survival in the 22 patients treated with second-line therapy. The MOS for the second-line cohort was 17 months (95% CI [6.5,26]). FIG. 9 is a subgroup analysis of the second-line cohort. In the second-line pemetrexed group (n=7), the MOS was 26 months (the 24 month survival rate was 62% with 3 of 7 patients still alive) with a median PFS of 8 months (95% CI [3,√]). In the second-line gemcitabine group (n=15), the MOS was 10 months (95% CI [4,21]) and the median PFS 3.5 months (95% CI [1.5,5.5]). MOS was not significantly associated with gender or age (data not shown).

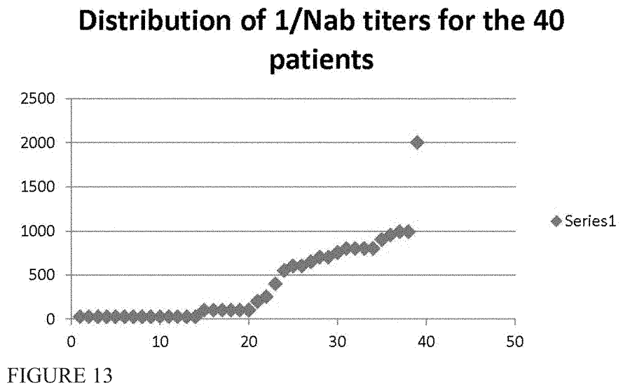

All potential patients were screened for baseline adenoviral Nab titers. Sixteen percent of the screened patients had titers above our pre-determined cut-off value of 1:2000 and were thus deemed ineligible. Of the 40 patients who participated in the trial, the median titer was 1:100; the distribution of titers is shown in FIG. 13.

### Biocorrelates

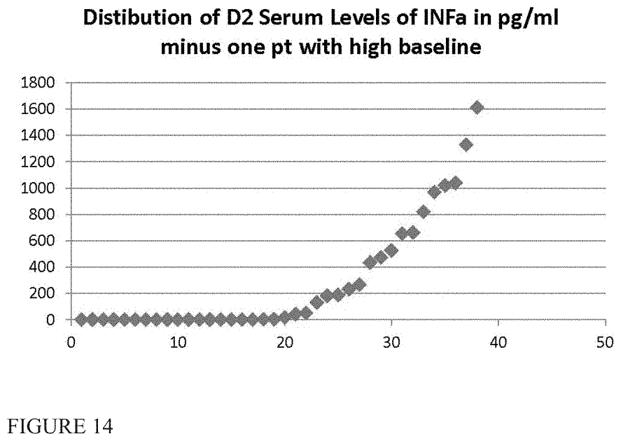

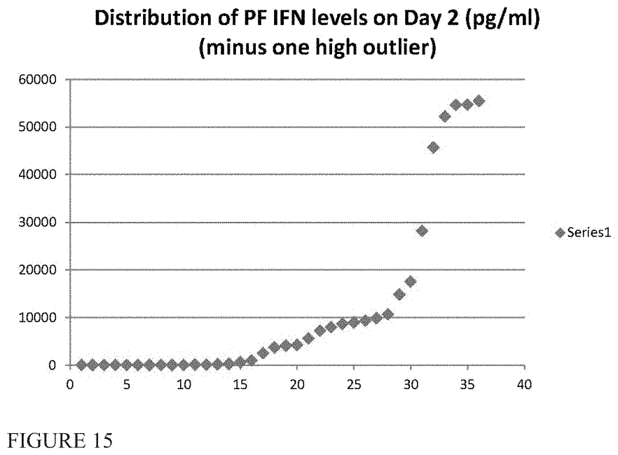

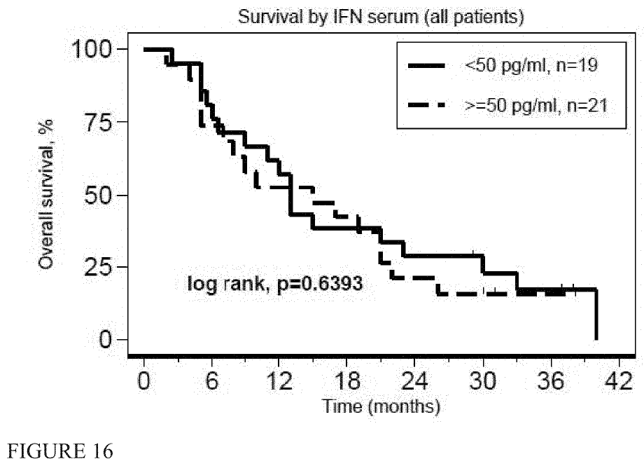

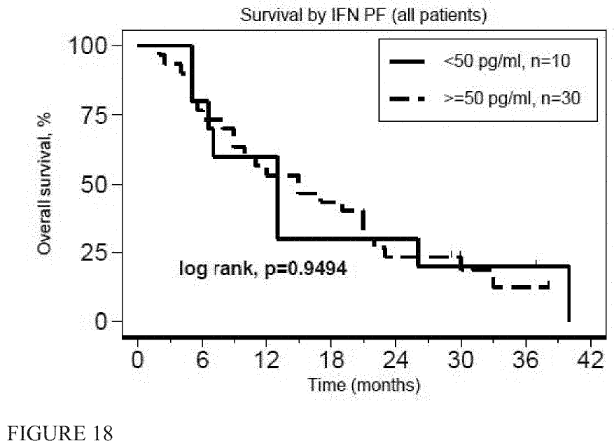

Serum levels of interferon-a were measured pre-vector infusion (Day 1) (FIG. 14). Serum IFN was undetectable or very low at baseline in 39 patients; one subject had high circulating levels before therapy (2100 pg/ml). Roughly half of the patients (n=21) had detectable levels of serum IFN (15 to 1608 pg/ml) on Day 2 after vector infusion. Of these patients, the median value was 470 pg/ml. Levels of IFNα in the pleural fluid or the pleural lavage were measured at baseline in 38 patients (FIG. 15). No patients had detectable baseline intrapleural IFNα. Pleural levels were much higher than seen in the serum after initial dosing. We saw no correlation of survival times with the serum (FIG. 16) or pleural (FIG. 18) interferon levels.

Expression of anti-tumor antibodies in the serum of post-treatment patients was available for analysis in 39 of the 40 patients. In 11 patients, we observed no changes in the number or intensity of anti-tumor immunoblot bands, in 14 there were minimal changes in tumor bands, and in the remaining 14 there were clear increases in anti-tumor bands. However, there were no significant differences in survival or in radiographic response rates among these groups (Table 8).

TABLE 8
Correlation of Antibody Response to
MOS or Radiographic Response

| Antibody | | MOS | Response |
|---|---|---|---|
| Response | N | (months) | (% change) |
| 0 | 11 | 13.0 | 0% |
| 1 | 14 | 14.0 | −12% |
| 2 | 14 | 12.5 | 7% |
| p value | | NS | NS |

We conducted flow cytometry from PBMC in 6 patients who had good responses (average survival=23.5 months) and compared results to 6 patients with poor responses (average survival=7.2 months) (Table 6). In previous studies, we had observed increases in the expression of the activation marker CD69 in natural killer (NK) cells after Ad.IFN administration in some patients, suggesting this could be a marker of systemic release of IFNα resulting in activation of the NK cells. Although we observed increases in the percent of NK cells and T cells expressing CD69 three days after Ad.IFNα instillation in the majority of patients, we detected no significant correlation with clinical responses (Table 9).

TABLE 9
CD8 T Cells and NK Cells Positive for CD69 Over Time

| Time | | |
|---|---|---|
| Baseline | D2 | D15 |

CD8 T Cells: % Positive for CD69

| | | | |
|---|---|---|---|
| Good Responders | 17.0 | 26.6 | 16.6 |
| | (IQR15.5-23.4) | (IQR: 23.2-44.9) | (IQR: 11.5-22.4) |
| Poor Responders | 11.4 | 22.1 | 14.9 |
| | (IQR: 9.3-20.9) | (IQR: 9.9-36.6) | (IQR: 10.5-44.8) |
| P Value: Good vs Bad | 0.235 | 0.235 | 1.000 |

NK Cells: % Positive for CD69

| | | | |
|---|---|---|---|
| Good Responders | 21.7 | 66.6* | 20.9 |
| | (IQR: 8.3-22.7) | (IQR: 42.1-79.0) | (IQR: 5.8-31.4) |
| Poor Responders | 17.5 | 33.3 | 22.7 |
| | (IQR: 11.1-24.6) | (IQR: 22.8-73.0) | (IQR: 13.2-44.6) |
| P Value: Good vs Bad | 1.000 | 0.298 | 0.92 |

*p = 0.03 vs Baseline

We observed no increases in the NK activation receptors NKp46, NKG2D, NKG2A and NKp30 (which had predicted response in a dendritic cell vaccine trial (21), nor changes in a CD8 T cell activation signature (CD38hi/HLA-DRhi and ki67hi/Bcl -2low) (22). We also noted no differences in baseline levels of CD4 T regulatory cells (CD4+/CD25+/FOXP3+cells) or changes in the induction of these cells. Increases in the expression of ICOS on CD4 cells have been associated with responses in melanoma patients treated with anti-CTLA4 antibody (23); however, we saw no significant changes in these markers (data not shown).

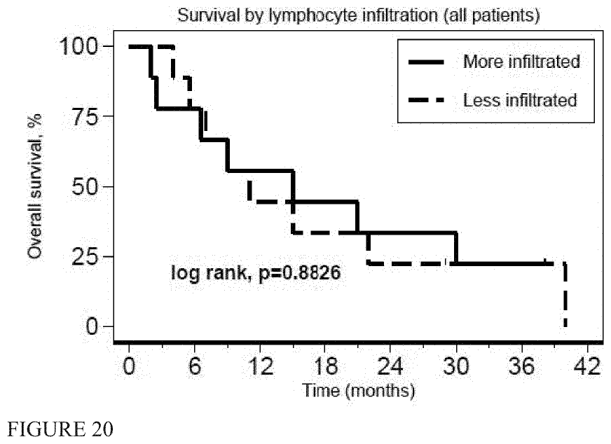

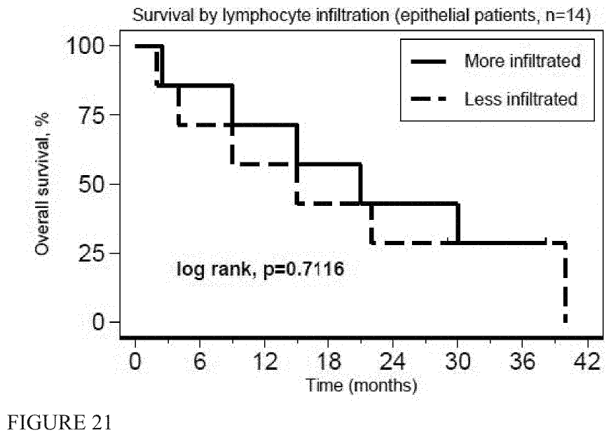

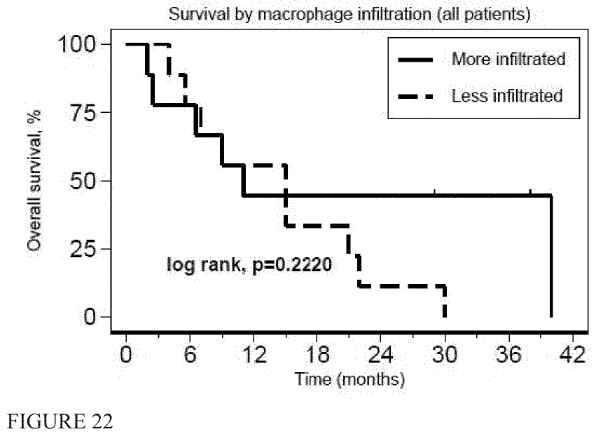

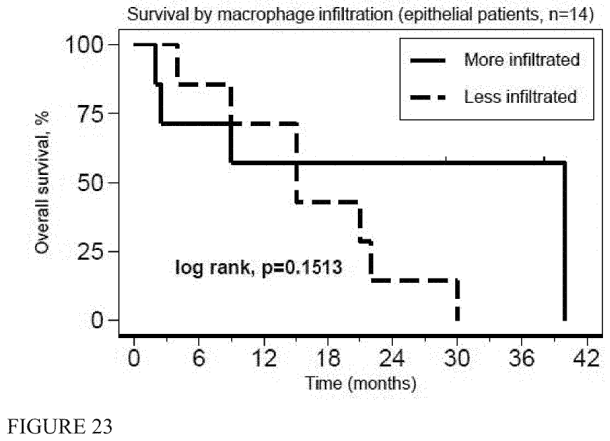

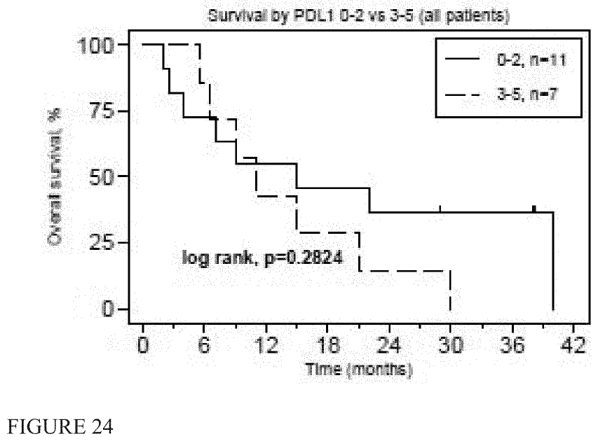

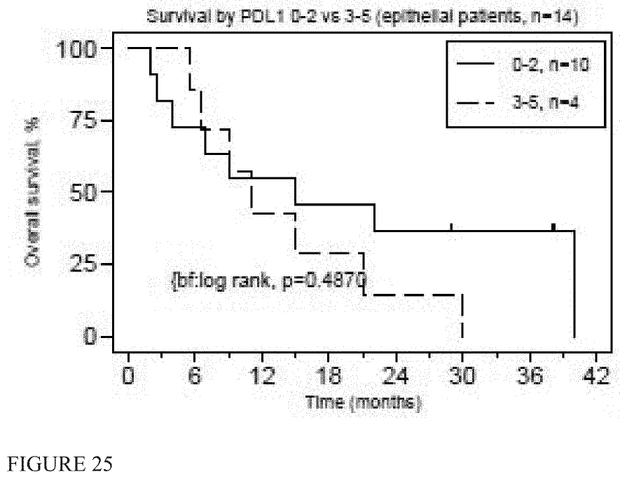

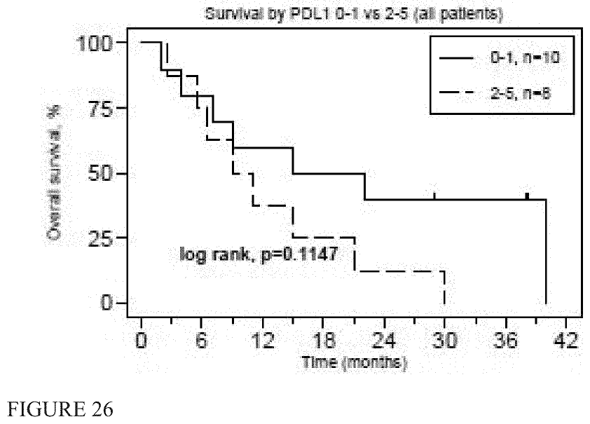

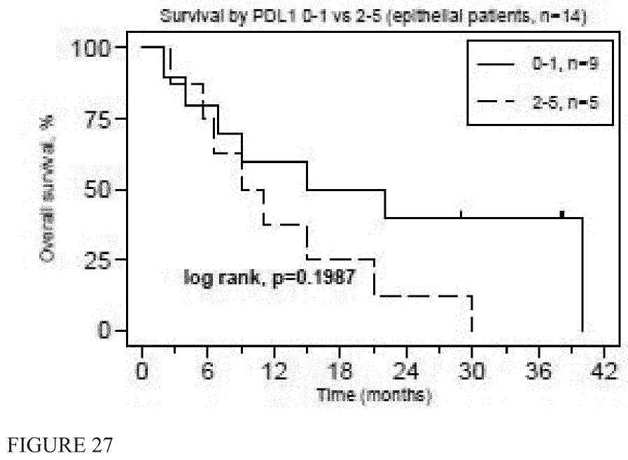

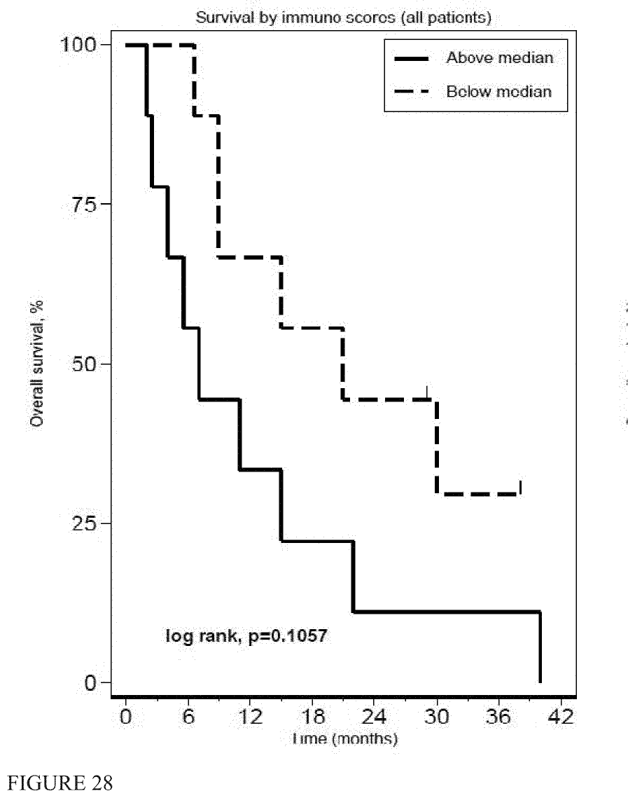

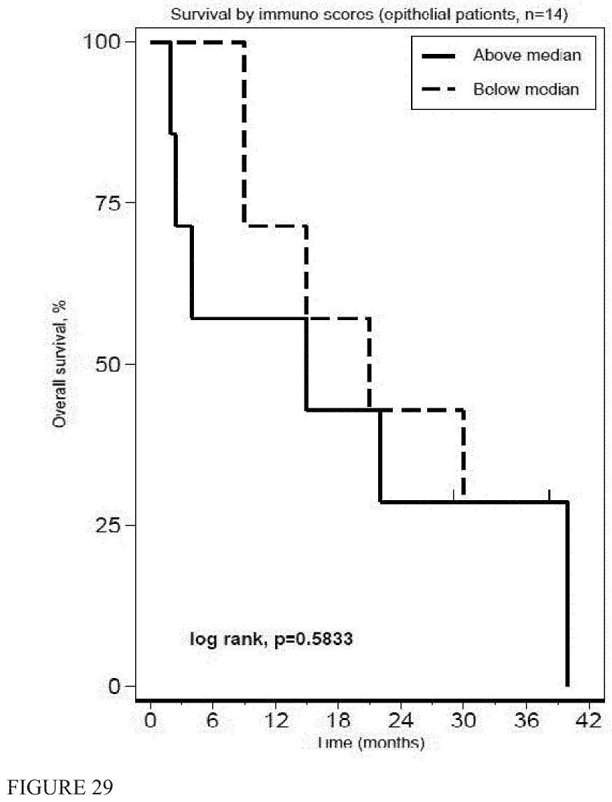

Finally, we investigated whether the “immunogenicity of the tumor microenvironment” could predict responses to immunotherapy (20, 24, 25) (using pathological material from pre-treatment biopsies available in 18 patients. Using immunohistochemistry (IHC), we noted no significant correlations with either the degree of lymphocyte (CD8 staining), or macrophage (CD68 staining) infiltration, nor expression of PD-L1 with survival (FIGS. 20-29). Slides were also used to produce RNA that was interrogated for 600 immune response-related genes using Nanostring® technology. We had information on 27 of the 61 PCR-validated genes from a recently published immune response gene signature (20). These markers are primarily T cell and interferon-induced genes (see Table 7). When the MPM specimens were ranked for intensity of expression of these genes, there was no significant correlation with survival (Supplemental FIG. 6).

### Discussion:

The rationale for this trial was to induce anti-tumor immune responses using an approach called “in situ vaccination,” a strategy where the tumor site itself is used as a target and becomes the source of antigen. We used the strong immune potentiating activity of an adenoviral vector expressing an activating transgene (interferon-α) to both induce immunogenic cell death and change the tumor microenvironment towards an immunostimulatory state. In addition, we attempted to further alter the tumor microenvironment by inhibiting the potent immunosuppressive molecule PGE2 (26) by administering a COX-2 inhibitor, celecoxib. Most cancer vaccines, however, require multiple administrations of antigen (“boosts”) for optimal efficacy (27, 28). Since the induction of neutralizing Ad antibodies prevented us from giving more than two, closely spaced doses of vector, we provided our “boost” by taking advantage of the observations that certain types of chemotherapy can cause cell death in an immunogenic context, thus stimulating a primed anti-tumor response (14-17). This is, therefore, one of the first clinical trials to formally employ a combination of in situ genetic immunotherapy and chemotherapy.

Our multi-pronged combination approach proved to be both feasible and safe in the majority of patients enrolled. In our study, 32/40 patients tolerated the combination therapy without evidence of serious adverse events; the majority of adverse events related to vector dosing were attributable to the initial dose; and 7 of the 8 patients who had serious adverse events after initial dosing were able to safely complete the course of celecoxib and chemotherapy. Only a single patient did not proceed with further chemotherapy dosing, and this was because of the esophagitis related to radiation therapy ,as previously described.

Based on our prior clinical trials involving repeated intrapleural dosing of recombinant Ad vectors expressing type I interferon genes (AdIFN beta and AdIFN alpha) (4-6), the majority of the observed toxicities were related to cytokine release syndrome secondary to the initial vector dose. In the current study, one of the primary outcome measures was the safety of sequential therapy with rAdIFN/celecoxib and chemotherapy. We did not believe that there would be substantial differences between the combination of one dose of rAdIFN and chemotherapy and that of two doses. As we had seen radiographic responses with single doses of AdIFN in prior Phase I clinical trials (4-6), it was reasonable from both a safety and efficacy perspective to allow patients to proceed in the study after only the initial rAdIFN dose.

In terms of clinical efficacy in first-line patients, our response rate, median PFS, MOS, and 1-year survival were similar to those previously reported in the literature with combination chemotherapy alone (See Table 4). However, our disease control rate was higher than reported with chemotherapy and there was a “tail” on the KM curve, representing a subset of patients with prolonged survival. This was observed despite the fact that only 11 of the 18 patients (61%) in our first-line cohort had the more favorable epithelial histology (a proportion lower than any of the reported trials (Table 4)). Although the numbers are small, the MOS in the epithelial, front-line group was 15 months versus only 8 months in the non-epithelial patients (p<0.05).

We believe that the lack of improvement in MOS seen in the front-line Pem/plat/rAdIFN group compared to historical controls was due to several factors, including: higher percentage of non-epithelioid tumors; pre-treatment with surgery and/or palliative radiotherapy; and selection of later stage patients as earlier stage patients with mesothelioma were shunted into concurrent trials of radical pleurectomy and photodynamic therapy at our institution. Surgery for mesothelioma was not nearly as well established in 2003 at the time of publication of the Vogelzang study, and therefore, many of the patients receiving chemotherapy in that trial would have been considered for surgical intervention at the present time.

Although the response rate and median PFS in second-line patients were similar to those from previously reported trials, the DCR and MOS were almost double those reported in similar second-line chemotherapy trials (Table 4). Similar to the front-line patients, we found a “survival tail” on the KM plots. Approximately 20% of second-line patients receiving gemcitabine-based chemotherapy were alive at 24 months, suggesting a prolonged immunologic phenomenon. Of special interest, however, was the finding that the 7 second-line patients undergoing re-treatment with pemetrexed had an especially impressive DCR of 100%, response rate of 28%, a PFS of 8 months, and a MOS of greater than 25 months. For comparison to this specific patient population, we were able to find data from three clinical trials (which included a total of 103 patients) that administered pemetrexed as second line therapy in patients who had previously responded to pemetrexed (Table 4). Although this group clearly has especially good response characteristics (with average reported response rates of 18%, PFS of 5.1 months, and MOS of 11.7 months), the patients in this trial responded to a much more impressive degree (see above).

The presence of patients with durable stable or slowly progressive disease resulting in prolonged survival has been observed in other immunotherapy trials (29). For example, recent studies using anti-CTLA antibodies have shown this pattern in melanoma and mesothelioma (30, 31). This pattern is consistent with observations that the effects of immunotherapy are frequently delayed, can show mixed patterns of response, and may not result in increased PFS or MOS while still engendering improved long-term survival rates (29, 32). Our long-term response data using radiographic measurements and SMRP levels, and the prolonged “stable disease” seen in many of our patients, are similar to other immunotherapy trials.

Despite our extensive investigations, we were unable to identify potential biomarkers that might provide prognostic and/or mechanistic information. This may be due to the fact that circulating cells or factors may poorly reflect processes within tumors; the implication being that the most useful biomarkers will need to be found from tumor biopsy specimens. This may be especially true for types of immunotherapy (such as ours) that generate polyclonal responses against unknown antigens, compared to vaccines where responses against a known specific antigen can be measured in the blood.

It is of interest to speculate on how Ad.IFN therapy might interface with checkpoint inhibitory blockade, an approach showing promise in mesothelioma (31). In contrast to checkpoint blockade therapy with anti-PD-1 or anti-PD-L1 antibodies, the expression of PD-L1 and the pre-existing immune signature of the tumor did not predict response to Ad.IFN. Given that in situ vaccination presumably works by inducing immune responses rather than simply amplifying existing endogenous immunity, Ad.IFN may be especially useful in those patients with minimal endogenous immune responses or low expression of PD-L1 and might be even more efficacious when combined with anti-CTLA4 or anti-PD1 antibodies. Preclinical studies to test these hypotheses are underway.

Since our study was relatively small, non-randomized, and conducted at a single center, it is important to recognize several potential limitations to the interpretation of the results. There is substantial heterogeneity in the clinical course of mesothelioma. A recently published registry study detailing the survival of patients MM posited that the MM population can be divided into two groups: one with a short survival time (9-12 months) and another small group that survives considerably longer (33). Any early stage clinical trial, such as ours, is subject to possible selection bias, including bias towards a good ECOG PS and a clinical status sufficient to tolerate access to the pleural cavity for intrapleural delivery of the Ad.IFN vector. Importantly, many of our patients received subsequent therapies with uncertain impact on ultimate survival [see Table 5].

Since our trial was non-randomized, our results can only be interpreted in the context of previously published studies with the presumption that the smaller second-line trials had the same sort of patient populations and similar biases as our trial. Using this admittedly imperfect comparator, a particularly interesting finding in our study was that patients with mesothelioma who received second-line chemotherapy (especially second-line pemetrexed) did extremely well when the chemotherapy was given subsequently to a priming protocol of immuno-gene therapy via Ad.IFN in situ vaccination plus targeted blockade of immune suppression by concomitant administration of celecoxib. As for the second-line pemetrexed patients, it is clear that this group fared better in terms of MOS than the second-line gemcitabine cohort (and, ironically, even better than first-line pemetrexed recipients). We were likely selecting patients with more favorable tumor biology given that they had a durable (at least 6-month) initial response to pemetrexed prior to disease progression. In addition, those patients who failed to respond to pem/platin and then received gemcitabine likely had a worse overall tumor biology than the treatment-naïve patients in the first-line cohort. Therefore, there were selection biases in both directions in the second-line arm of the trial. Perhaps most importantly, these same biases are present in every second-line chemotherapy trial in mesothelioma, and our reported overall survival rates in second line are superior to prior reports of retreatment with pemetrexed as well as with gemcitabine (see Supplemental Table 1).

These results raise several interesting, but as yet unanswered questions: 1) why did second-line patients respond so much better than first-line recipients?; 2) why do patients receiving a repeat course of pemetrexed perform better than those on the second line gemcitabine? 3) Is it possible that the patients who initially responded to pemetrexed and were then retreated have been pre-selected as long-term stable disease?; and 4), if the immune response is to be credited with the difference in survival, then why are there no markers of immune responsiveness that correlate with this outcome? A biopsy subsequent to therapy would be have been helpful in determining intratumoral markers of immune responsiveness, but was not included in this clinical protocol. Hopefully, some of these questions can be answered in future studies.

We do not yet know the optimal chemotherapy regimen for “immunological priming” in mesothelioma. The potential role of chemotherapy in combination with immunotherapy is multifold, and includes: tumor cell death resulting in presentation of tumor neo-antigens to dendritic cells; decreased numbers of myeloid-derived suppressor cells and regulatory T cells; overall T cell depletion allowing increased space in the existing T-cell repertoire for tumor-specific cytotoxic T cells; and increased T cell trafficking into the tumor microenvironment (14-17). Our laboratory has spent considerable effort in evaluating these characteristics of both pemetrexed and gemcitabine in syngeneic, immunocompetent murine models of malignant mesothelioma, and has demonstrated significant synergy for both chemotherapy agents with murine versions of rAdIFN. We selected pemetrexed for first line therapy in this clinical trial in large part because of its accepted role as the standard of care chemotherapy agent for front-line therapy in mesothelioma; gemcitabine is a well-accepted second-line agent for mesothelioma. It is possible, however, that gemcitabine may be a more effective agent to use in front-line therapy with rAdIFN than pemetrexed, and we hope to answer this question in future human clinical trials.

In conclusion, our study shows that the combination of intrapleural Ad.IFN-α2b vector, celecoxib, and systemic chemotherapy proved to be safe, feasible, and well-tolerated in MPM patients. Disease control and survival rates observed in this study, especially in the second-line therapy compared favorably with historical data. Obviously, the value of our approach needs to be validated with a larger, multi-center randomized clinical trial. Such a study is being planned in the second-line setting where no therapy has yet been shown to enhance survival in patients with mesothelioma.

### Supplemental Methods

### Definitions of Dose Limiting Toxicity

The primary endpoint of the clinical trial was to identify new toxicities and the safety of two doses of intrapleural AdIFN in combination with standard of care chemotherapy for MPM. Enrolled subjects were evaluated for dose-limiting toxicity (DLT) from the start of celecoxib and the first dose of Ad.IFN to 14 days after the first round of chemotherapy (approximately Day 30). Dose limiting toxicities (DLTs) were defined as any of the following treatment-related adverse events (AEs) as per the Common Terminology Criteria for Adverse Events (CTCAE v.3.0) adopted by the National Cancer Institute:

Any Grade 4 toxicity (except isolated Grade 4 lymphopenia lasting ≤7 days after the last dose of AdIFN).

Grade 3 hypotension, disseminated intravascular coagulation (DIC) or allergic reaction/hypersensitivity.

Grade 3 non-hematologic toxicity persisting for >7 days except for cytokine release syndrome (CRS) within 6-48 hours after AdIFN dosing.

Persistent CRS starting within 48-72 hours of dosing and lasting up to 10 days after last dose of AdIFN.

Grade 3 hematologic toxicity persisting for >7 days after last vector dose (except isolated lymphopenia)

If a DLT occurred during the infusion, AdIFN administration was stopped, and no further study drug was to be administered. If a DLT occurred between Day 1 and Day 3, the second dose of study drug would not be administered.

The protocol stopping rules stipulate that if two (2) DLTs occurred within the first treatment group, enrollment in the study was to be halted pending a review of the data and discussion with the FDA and IRB about de-escalation.

In addition, the protocol specified that subjects may be withdrawn from the study prior to the expected completion if, among other events, the subject experiences a DLT or serious adverse event, or if a chemotherapy cycle is delayed more that 3 weeks from scheduled cycle due to lack of resolution of toxicities.

### Procedures

### Immunoblotting

To detect induced humoral responses against tumor antigens, we performed immunoblotting against purified mesothelin and extracts from allogeneic mesothelioma cell lines. Purified mesothelin was purchased from Raybiotech (Norcross, Ga.). Cell lines were derived from patient pleural fluid samples from previous clinical trials and were grown in culture as previously described (Sterman D H, Reico A, Haas A R, et al. A phase I trial of repeated Intrapleural adenoviral-mediated interferon-beta gene transfer for mesothelioma and metastatic pleural effusion. Mol Ther 2010; 18: 852-60). Extracts from cells or purified proteins were prepared and immunoblotted with patient serum (diluted at 1:1500) from time points before treatment, and 6 weeks after treatment as previously described (Sterman et al., 2010). Multiple exposures were obtained and comparisons were made on the exposures in which the major bands detected on pre-treatment blots were of equal intensity in post-treatment blots.

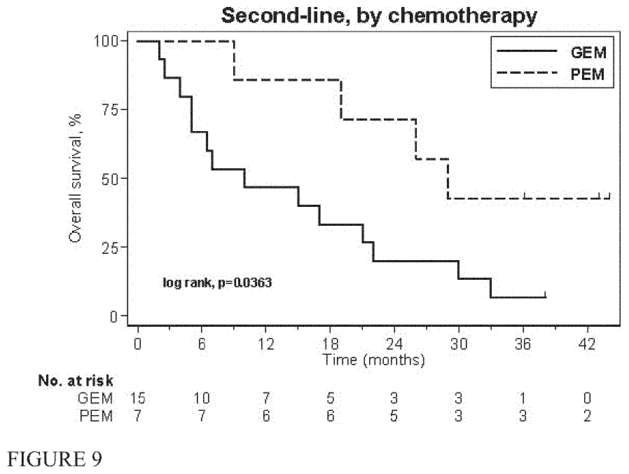

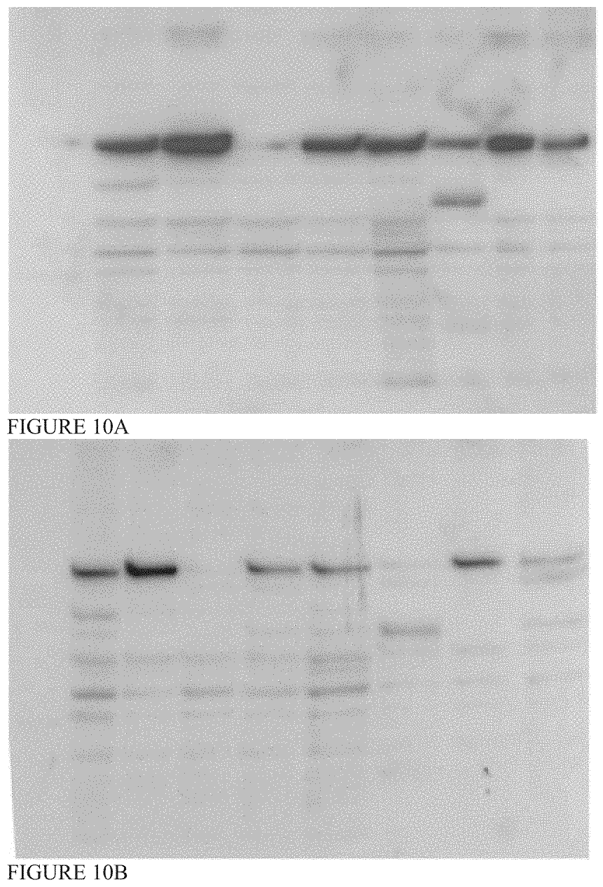

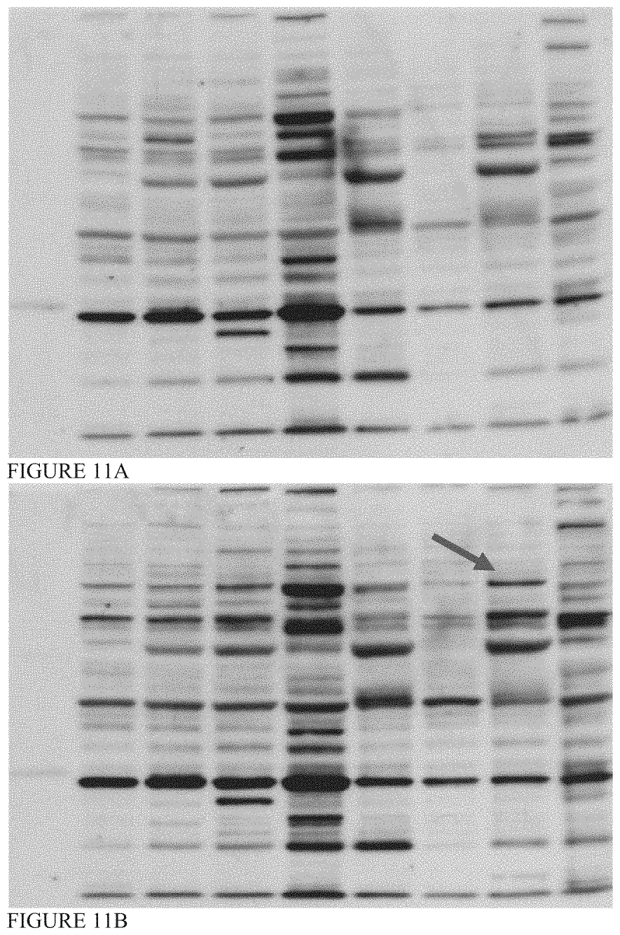

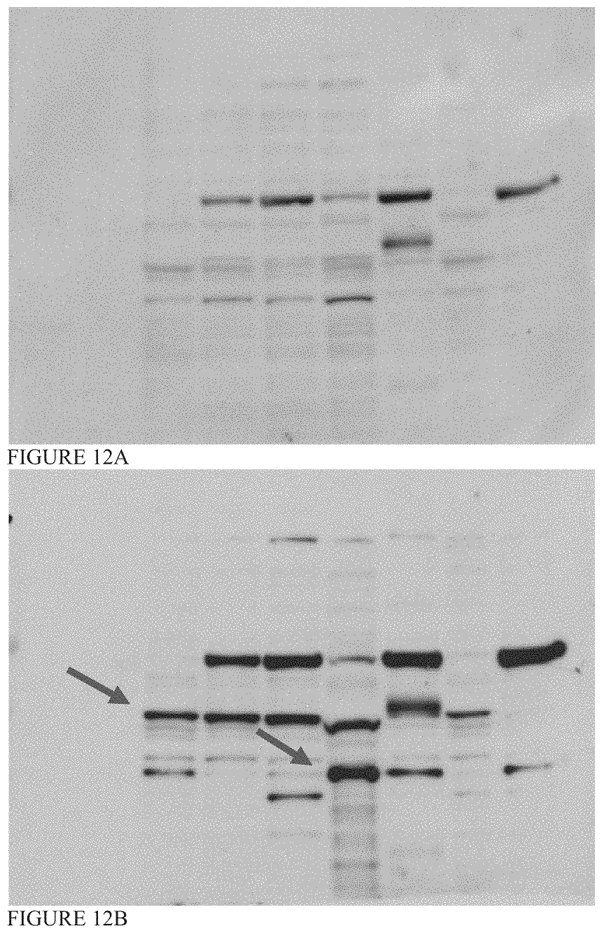

Two independent, blinded observers visually scanned each blot to detect new bands or bands that appeared markedly increased in the post-treatment serum and came to a consensus score. The blots were semi-quantitatively scored as follows: 0=no change in any bands; 1=minimal changes (i.e. increased intensity in one or two bands); 2=clear increases in >2 bands or appearance of new bands. A sample showing each scoring category is shown in FIGS. 10-12.

### Flow Cytometry

Cryopreserved peripheral blood mononuclear cells (PBMC) collected prior to treatment, 2 days after Ad.INF instillation (before the second dose) and Day 15 days after the first vector dose (before chemotherapy) were thawed, and natural killer cell (NK) and T cell subsets and their activation status, were assessed with mAbs against CD3, CD4, CD25, FoxP3, CD8, CD56, CD16, CD69, CD38, HLA-DR, Ki67, Bc12, ICOS, NKG2D, NKG2A, and NKp30. All mAbs were from BD Biosciences (San Diego, Calif.) and R&D systems (Minneapolis, Minn., USA). PBMCs from a set of six patients who responded the therapy and 6 patients who did not were studied (Table 5).

Details of the cell preparation and staining have been previously published (Stevenson J P, Kindler H L, Papasavvas E, et al. Immunological effects of anti-transforming growth factor-beta (TGF-beta) antibody GC1008 in cancer patients with malignant pleural mesothelioma (MPM). Oncoimmunology 2013; 8 :e26218). Analysis was done by collecting 100,000 live lymphocytes (defined by size and granularity in FSC and SSC). Dead cells were excluded by manual gating in FSC/SSC. Detection thresholds were set according to isotype-matched negative controls. Results were expressed as Mean Fluorescent Intensity (MFI) and percent (%) of lymphocytes, NK cells (Lin3-CD56dimCD16+), CD3+CD4+ or CD3+CD8+ T cells. Data analysis was performed using FloJo software (Tree Star, San Carlos, Calif.).

### Immunohistochemistry

Formalin-fixed paraffin-embedded sections from original surgical biopsies or previous surgery were available from 18 patients. After deparaffinization and antigen retrieval, these were stained by the Penn Cancer Center Pathology Core for T cells (using anti-CD8 antibody) and macrophages (using anti-CD68 antibody). Sections were also stained for anti-PD-L1 by Merck using a proprietary antibody (clone 22C3). Sections were scored for quantity of PD-L1 expression by a pathologist in a blinded fashion on a 0 to five scale: 0=negative, 1=trace/rare, 2=low, 3=moderate, 4=high, and 5=very high.

Tissue sections were also used for RNA analysis using Nanostring analysis. Prior to making the cell lysate or isolating the RNA, tissue sections were deparaffinized in xylene for 3×5 min and then rehydrated by immersing consecutively in 100% ethanol for 2×2 min, 95% ethanol for 2 min, 70% ethanol for 2 min and then immersed in dH2O until ready to be processed. Tissue was lysed on the slide by adding 10-50u1 of PKD buffer (Qiagen catalog #73504). Tissue was scraped from the slide and transferred to a 1.5 ml eppendorf tube. Proteinase K (Roche Prot-K catalog # 03115836001) was added at no more than 10% final volume and the RNA lysate was incubated for 15 min at 55° C. and then 15 min at 80° C. The RNA lysate or total RNA was stored at −80° C. until gene expression profiling was performed using the NanoString nCounter™ system. 50 ng of cellular lysate or total RNA per sample, in a final volume of 5 ul, was mixed with a

### Flow Cytometry Statistics

Data were described as medians, 25th and 75th percentiles. Comparisons between responders (n=6) and non-responders (n=6) at each time point were done using Wilcoxon Kruskal-Wallis tests (Rank Sums). Differences between time points for all patients (n=12) were tested using Wilcoxon Signed-Rank or paired t-tests depending on data distribution, while differences between time points in responders (n=6) and in non-responders (n=6) were tested using Wilcoxon Signed-Rank. p values that were less than 0.05 were considered statistically significant. All statistics used JMP

### Summary

Given our specific disclosure, the rtisan can readily devise alternative iterations. For example, while we use rAd-IFN, other agents are known to induce interferon. Similarly, while we use Celecoxib, the art teaches equivalent COX-2 enzyme inhibitors. We thus intend the scope of our patent to be defined not by our specific examples taught here, but by our appended legal claims and permissible equivalents thereto.

In the appended legal claims, we use the term “epithelioid” to describe cancer with a purely epithelioid histology, and with a biphasic histology having at least about 90% epithelioid histology.

### REFERENCES

1. Ai J, Stevenson J P. Current issues in malignant pleural mesothelioma evaluation and management. Oncologist 2014; 19:975-84.
2. Zucali P A, Simonelli M, Michetti G, Tiseo M, Ceresoli G L, Collová E, et al. Second-line chemotherapy in malignant pleural mesothemioma: Results of a retrospective multicenter survey. Lung Cancer 2012; 75:360-67.
3. Vachani A, Moon E, Wakeam E, Albelda S M. Gene Therapy for Mesothelioma and Lung Cancer. Am J Respir Cell Mol Biol 2010; 42:385-93.
4. Sterman D H, Recio A, Carroll R G, Gillespie C T, Haas A, Vachani A, et al. A phase I clinical trial of single-dose Intrapleural INF-beta gene transfer for malignant pleural mesothelioma and metastatic pleural effusions: high rate of antitumor immune responses. Clin Cancer Res 2007; 13:4456-66.
5. Sterman D H, Recio A, Haas A R, Vachani A, Katz S I, Gillespie C T, et al. A phase I trial of repeated Intrapleural adenoviral-mediated interferon-beta gene transfer for mesothelioma and metastatic pleural effusion. Mol Ther 2010; 18:852-60.
6. Sterman D H, Haas A, Moon E, Recio A, Schwed D, Vachani A, et al. A trial of intrapleural adenoviral-mediated Interferon-α2b gene transfer for malignant pleural mesothelioma. Am J Respir Crit Care Med 2011; 184:1395-9.
7. Ferrantini M, Capone I, Belardelli F. Interferon-alpha and cancer: Mechanisms of action and new perspectives of clinical use. Biochimie 2007; 89:884-93.
8. Boutin C, Nussbaum E, Monnet I, Bignon J, Vanderschueren R, Guerin J C, et al. Pleural 1 treatment with recombinant gamma-interferon in early stage malignant pleural mesothelioma. Cancer. 1994; 74:2460-7.
9. Arico E, Belardelli F. Interferon-alpha as antiviral and antitumor vaccine adjuvants: Mechanisms of action and response signature. J Interferon Cytokine Res 2012; 32:235-47.
10. Majhen D, Calderon H, Chandra N, Fajardo C A, Rajan A, Alemany R, et al. Adenovirus-Based vaccines for fighting infectious diseases and cancer: progress in the field. Hum Gene Ther 2014; 25:301-17.
11. Dinney C P, Fisher M B, Navai N, O'Donnell M A, Cutler D, Abraham A, et al. Phase I trial of intravesical recombinant adenovirus mediated interferon-α2b formulated in Syn3 for bacillus Calmette-Guerin failures in nonmuscle invasive bladder cancer. J Urol 2013; 190:850-6.
12. Haas A R, Sun J, Vachani A, Wallace A F, Silverberg M, Kapoor V, et al. Cyclooxygenase-2 inhibition augments efficacy of a cancer vaccine. Clin Cancer Res 2006; 12:214-22.
13. Fridlender Z G, Sun J, Singhal S, Kapoor V, Cheng G, Suzuki E, et al. Chemotherapy delivered after viral immune-gene therapy augments anti-tumor efficacy via multiple immune-mediated mechanisms. Mol Ther 2010; 18:1947-59.
14. Lake R A, Robinson B W S. Immunotherapy and chemotherapy—a practical partnership. Nat Rev Cancer 2005; 5:397-405.
15. McCoy M J, Nowak A K, Lake R A. Chemoimmunotherapy: an emerging strategy for the treatment of malignant mesothelioma. Tissue Antigens 2009; 74:1-10.
16. Zitvogel L, Apetoh L, Ghiringhelli, Kroemer G. Immunological aspects of cancer chemotherapy. Nat Rev Immunol 2008; 8:59-73.
17. de Biasi A R, Vliiena-Vargas J, Adusumilli P S. Cisplatin-induced antitumor immunomodulation: a review of preclinical and clinical evidence. Clin Cancer Res 2014; 20:5384-91.
18. Byrne M J, Nowak A K. Modified RECIST criteria for assessment of response in malignant pleural mesothelioma. Ann Oncol 2004; 15:257-60.
19. Stevenson J P, Kindler H L, Papasavvas E, Sun J, Jacobs-Small M, Hull J, et al. Immunological effects of anti-transforming growth factor-beta (TGF-beta) antibody GC1008 in cancer patients with malignant pleural mesothelioma (MPM). Oncoimmunology 2013; 8:e26218.
21. Osada T, Clay T, Hobeika A, Lyerly H K, Morse M A. NK cell activation by dendritic cell vaccine: a mechanism of action for clinical activity. Cancer Immunol Immunother 2006; 55:1122-31.
22. Miller J D, van der Most R G, Akondy R S, Glidewell J T, Albott S, Masopust D, et al. Human effector and memory CD8+ T cell responses to smallpox and yellow fever vaccines. Immunity 2008; 28:710-22.
23. Ng Tang D, Shen Y, Sun J, Wen S, Wolchok J D, Yuan J, et al. Increased frequency of ICOS+ CD4 T cells as a pharmacodynamic biomarker for anti-CTLA-4 therapy. Cancer Immunol Res 2013:1:229-34.
20 Ulloa-Montoya F, Louahed J, Dizier B, Gruselle O, Spiessens B, Lehmann F F, et al. Predictive gene signature in MAGE-A3 antigen-specific cancer immunotherapy. J Clin Oncol 2013; 31:2388-95.
24. Ji R R, Chasalow S D, Wang L, Hamid O, Schmidt H, Cogswell J, et al. An immune-active tumor microenvironment favors clinical response to ipilimumab. Cancer Immunol Immunother 2012; 61:1019-31.
25. Wang E, Bedognetti D, Marincola F M. Prediction of response to anticancer immunotherapy using gene signatures. J Clin Oncol 2013; 31:2369-71.
26. Nakanishi M, Rosenberg D W. Multifaceted roles of PGE2 in inflammation and cancer. Semin Immunopathol 2013; 35:123-37.
27. Melero I, Gaudernack G, Gerritsen W, Huber C, Parmiani G, Scholl S, et al. Therapeutic vaccines for cancer: an overview of clinical trials. Nat Rev Clin Oncol 2014; 11:509-24.
28. Palena C and Schlom J. Vaccines against human carcinoma: strategies to improve antitumor immune responses. J Biomed Biotechnol. 2010; 2010:380697.
29. Madan R A, Gulley J L, Fojo T, Dahut W L. Therapeutic cancer vaccines in prostate cancer: the paradox of improved survival without changes in time to progression. Oncologist. 2010; 15:969-75.
30. Schadendorf D, Hodi F S, Robert C, Weber J S, Margolin K, Hamid O, et al. Pooled analysis of long-term survival data from Phase II and Phase III trials of ipilimumab in unresectable or metastatic melanoma. J Clin Oncol 2015; 33:1889-94.
31. Calabrò L, Morra A, Fonsatti E, Cutaia O, Amato G, Giannarelli D, et al. Tremelimumab for patients with chemotherapy-resistant advanced malignant mesothelioma: an open-label, single-arm, phase 2 trial. Lancet Oncol 2013; 14:1104-11.
32. Hoos A, Eggermont A M, Janetzki S, Hodi F S, Ibrahim R, Anderson A, et al. Improved endpoints for cancer immunotherapy trials. J Natl Cancer Inst 2010; 102:1388-97.
33. Linton A, Pavlakis N, O'Connell R, Soeberg M, Kao S, Clarke S, et al. Factors associated with survival in a large series of patients with malignant pleural mesothelioma in New South Wales. Br J Cancer 2014; 111:1860-1869.

we claim:

1. A method of treating a human patient comprising:
a. diagnosing in said patient cancer; and then b. treating said patient with a first-line treatment regimen comprising treatment selected from the group consisting of: an anti-folate and platin combination regimen; an anti-folate and platin and bevacizumab combination regimen; a programmed cell death-1 (PD-1) inhibitor; a programmed cell death ligand-1 (PD-L1) inhibitor; and a cytotoxic T-lymphocyte-associated protein 4 (CTLA-4) inhibitor and PD-1 or PD-L1 inhibitor combination regimen; and then
c. diagnosing in said patient cancer resistant to or recurrent after said first-line treatment regimen; and then
d. treating said patient with an agent which induces the expression of interferon, in an amount effective to induce interferon expression in a human; and then
e. treating said patient with a second-line treatment regimen comprising treatment selected from the group consisting of: pemetrexed, gemcitabine, cisplatin and carboplatin.

2. The method of claim 1, wherein the second-line treatment regimen comprises pemetrexed.

3. The method of claim 1, wherein the second-line treatment regimen comprises gemcitabine.

4. The method of claim 1, wherein the agent which induces the expression of interferon comprises antigen.

5. The method of claim 4, wherein the antigen comprises viral antigen.

6. The method of claim 5, where the viral antigen comprises antigenic virus.

7. The method of claim 6, where the antigenic virus comprises a transgene encoding human interferon.

8. The method of claim 7, where said antigenic virus comprises rAd.IFN.

9. The method of claim 8, where said rAd.IFN is provided in a dose of about 3×10¹¹ viral particles, delivered intrapleurally.

10. The method of claim 1, said second line treatment regimen further comprising treatment selected from the group consisting of: bevacizumab; a programmed cell death-1 (PD-1) inhibitor; a programmed cell death ligand-1 (PD-L1) inhibitor; and a cytotoxic T-lymphocyte-associated protein 4 (CTLA-4) inhibitor.

11. The method of claim 1, said second line treatment regimen further comprising treatment with celecoxib.

12. The method of claim 1, wherein said cancer resistant to or recurrent after said first-line treatment regimen comprises epithelioid cancer.

13. The method of claim 12, wherein said epithelioid cancer comprises malignant pleural mesothelioma.

14. A method of treating a human patient comprising:
a. diagnosing in said patient cancer; and then
b. treating said patient with a first-line cancer treatment regimen; and then
c. diagnosing in said patient said cancer, said cancer resistant to or recurrent after said first-line cancer treatment regimen; and then
d. treating said patient with an agent which induces the expression of interferon, in an amount effective to induce interferon expression in a human; and
e. treating said patient with an agent which inhibits an inhibitory human immune system checkpoint.

15. The method of claim 14, wherein said cancer comprises cancer selected from the group consisting of: bladder cancer, non-small cell lung carcinoma and mesothelioma.

16. The method of claim 15, wherein said cancer comprises bladder cancer.

17. The method of claim 14, further comprising:
f. treating said patient with a cytotoxic selected from the group consisting of: pemetrexed, gemcitabine and platinum-based cytotoxic.

18. The method of claim 17, wherein the cytotoxic comprises pemetrexed.

19. The method of claim 17, wherein the cytotoxic comprises platinum-based cytotoxic.

20. The method of claim 14, wherein the agent which inhibits an inhibitory human immune system checkpoint is selected from the group consisting of: pembrolizumab, nivolumab and ipilimumab.

21. A method of treating a human patient comprising:
a. diagnosing in said patient cancer; and then
b. treating said patient with an agent which induces the expression of interferon, in an amount effective to induce interferon expression in a human; and
c. treating said patient with an agent which inhibits an inhibitory human immune system checkpoint.

22. The method of claim 21, the agent which inhibits an inhibitory human immune system checkpoint selected from the group consisting of: pembrolizumab, nivolumab and ipilimumab.
